(19) United States
(12) Patent Application Publication
(10) Pub. No.: US 2023/0290039 A1
(43) Pub. Date: Sep. 14, 2023

(54) 3D Graph Visualizations to Reveal Features of Disease

(71) Applicant: Octave Bioscience, Inc., Menlo Park, CA (US)

(72) Inventors: David A. Hughes, Seattle, WA (US); Anisha Keshavan, Encinitas, CA (US); Kelly Michelle Leyden, Redwood City, CA (US); Erwan Frederic Pierre Rivet, Menlo Park, CA (US); William A. Hagstrom, Menlo Park, CA (US)

(21) Appl. No.: 18/023,813

(22) PCT Filed: Aug. 31, 2021
(86) PCT No.: PCT/US21/48442

§ 371 Date: Feb. 28, 2023

Related U.S. Application Data
(60) Provisional application No. 63/073,022, filed on Sep. 1, 2020.

(51) Int. Cl. G06T 15/08; G06T 7/00; G16H 30/40

(52) U.S. Cl. CPC G06T 15/08; G06T 7/0014; G16H 30/40; G06T 2210/41; G06T 2207/30096; G06T 2207/10088; G06T 2207/30016

(57) ABSTRACT

Three dimensional (3D) graph structures are constructed from images captured from subjects. The 3D graph structures are composed of nodes which can be queried to identify presence of anatomical abnormalities, such as multiple sclerosis lesions. As additional images are captured from the subjects over time, 3D graph structures are efficiently updated and analyzed, which reveals the topology and temporal nature of multiple sclerosis disease by exposing novel structural features of the brain through representation of data as interactive 3D projections.

### CROSS REFERENCE TO RELATED APPLICATIONS

This application claims the benefit of and priority to U.S. Provisional Patent Application No. 63/073,022 filed Sep. 1, 2020, the entire disclosure of which is hereby incorporated by reference in its entirety for all purposes. All references, issued patents and patent applications cited within the body of the instant specification are hereby incorporated by reference in their entirety, for all purposes.

### BACKGROUND

Conventional methods for characterizing multiple sclerosis disease involves capturing magnetic resonance imaging (MRI) data which is then analyzed by a trained medical expert (e.g., a radiologist) for purposes of diagnosing or characterizing the disease. However, conclusions by different trained medical experts may differ, thereby rendering the diagnosis or characterization inconclusive. There is a need for novel visualization of neuroimaging data which can lead to clinical insights and new imaging analysis capabilities.

### SUMMARY

Disclosed herein are methods, non-transitory computer-readable media, and systems for constructing 3D graph structures using images (e.g., magnetic resonance imaging (MRI) data). The 3D graph structures are composed of nodes and edges which are queried to identify presence of anatomical abnormalities, such as multiple sclerosis lesions. Thus, implementation of the 3D graph reveals the topology and temporal nature of multiple sclerosis disease, by exposing novel structural features of the brain through representation of data as interactive 3D projections.

Embodiments of the disclosed invention achieve at least two improvements. First, the implementation of 3D graphs enables improved visualization and understanding of diseases such as multiple sclerosis. Typically, trained experts (e.g., a neurologist) obtains MRI scans and manually identifies presence of lesions on 2D planes to draw conclusions of the disease, which can be cumbersome and time consuming.

Second, the methods described herein can be implemented by a computational system in a manner that reduces the consumption of resources. Namely, 3D graphs as well as node neighborhoods identifying the presence of lesions can be stored such that at a subsequent time, they need not be regenerated, which can be resource intensive and time-intensive. For example, a 3D graph and a lesion node neighborhood can be retrieved when additional MRI images are captured, such that the 3D graph and lesion node neighborhood can be updated, thereby revealing the topological features and temporal changes of a lesion. Thus, 3D graphs can be stored and continuously updated over time to build a personalized 3D graph representation for the subject without needing to re-analyze the raw images (e.g., MRI images).

Disclosed herein is a method comprising: obtaining a set of images captured from an individual, the set of images comprising an anatomical abnormality; generating a three dimensional (3D) graph using the set of images, the 3D graph comprising a plurality of nodes representing voxels and the anatomical abnormality; establishing a seed node in the 3D graph indicative of a presence of the anatomical abnormality, the seed node defined by an initial voxel coordinate; defining a node neighborhood comprising the seed node indicative of a 3D volume of the anatomical abnormality by: iteratively interrogating one or more adjacent nodes for inclusion or exclusion from the node neighborhood, wherein the interrogation of each of the one or more adjacent nodes is based on an intensity value of the adjacent node and an anatomical location of the adjacent node; generating a representation of the 3D volume of the anatomical abnormality; and storing at least the representation of the 3D volume of the anatomical abnormality. In various embodiments, methods disclosed herein further comprise: obtaining a second set of images captured from the individual, the second set of images further comprising the anatomical abnormality; generating a second three dimensional (3D) graph using the second set of images, the 3D graph comprising a plurality of nodes representing voxels and the anatomical abnormality; establishing a seed node in the second 3D graph indicative of a presence of the anatomical abnormality in the second 3D graph, the seed node defined by an initial voxel coordinate; defining a node neighborhood comprising the seed node indicative of a 3D volume of the anatomical abnormality by: iteratively interrogating one or more adjacent nodes for inclusion or exclusion from the node neighborhood, wherein the interrogation of each of the one or more adjacent nodes is based on an intensity value of the adjacent node and an anatomical location of the adjacent node; generating a second representation of the 3D volume of the anatomical abnormality; retrieving at least the stored representation of the 3D volume of the anatomical abnormality; characterizing the anatomical abnormality by comparing the stored representation of the 3D volume of the anatomical abnormality to the second representation of the 3D volume of the anatomical abnormality.

In various embodiments, interrogation of the one or more adjacent nodes of the 3D graph or of the second 3D graph comprises: retrieving a threshold value previously determined for the anatomical location; comparing the intensity value of the adjacent node to the retrieved threshold value. In various embodiments, methods disclosed herein further comprise: responsive to determining that the intensity value of the adjacent node exceeds the retrieved threshold value, including the adjacent node in the node neighborhood. In various embodiments, methods disclosed herein further comprise: responsive to determining that the intensity value of the adjacent node is less than the retrieved threshold value, excluding the adjacent node from the node neighborhood.

In various embodiments, interrogation of the one or more adjacent nodes of the 3D graph or of the second 3D graph comprises: determining whether the anatomical location of the adjacent pixel differs from an anatomical location of the seed node. In various embodiments, methods disclosed herein further comprise: responsive to determining that the anatomical location of the adjacent node does not differ from the anatomical location of the seed node, including the adjacent node in the node neighborhood. In various embodiments, methods disclose herein further comprise: responsive to determining that the anatomical location of the adjacent node differs from the anatomical location of the seed node, excluding the adjacent node in the node neighborhood.

In various embodiments, the anatomical location of the node is a neuroanatomical location of the node. In various embodiments, the neuroanatomical location of the node comprises one or more of 3rd Ventricle, 4th Ventricle, 5th Ventricle, Amygdala, Anterior Cingulate, Anterior Middle Frontal, Brainstem, Caudal Anterior Cingulate, Caudate, Cerebellar Gray Matter, Cerebellar White Matter, Cerebral White Matter, Cerebral WM Hypointensities, Cortical Gray Matter, Cuneus, Entorhinal Cortex, Frontal Pole, Fusiform, Hippocampus, Inferior Frontal, Inferior Lateral Ventricles, Inferior Parietal, Inferior Temporal, Insula, Isthmus Cingulate, Lateral Occipital, Lateral Orbitofrontal, Lingual, Medial Occipital, Medial Orbitofrontal, Medial Parietal, Middle Frontal, Middle Temporal, Nucleus Accumbens, Pallidum, Paracentral, Parahippocampal, Pars Opercularis, Pars Orbitalis, Pars Triangularis, Pericalcarine, Posterior Cingulate, Posterior Superior Temporal Sulcus, Premotor, Primary Motor, Primary Sensory, Putamen, Rostral Anterior Cingulate, Superior Frontal, Superior Lateral Ventricles, Superior Parietal, Superior Temporal, Supramarginal, Temporal Pole, Thalamus, Transverse Temporal, Transverse Temporal+Superior Temporal, Ventral Diencephalon, Whole Brain, Intracranial Volume, Forebrain Parenchyma, Ventricles, Cerebellum, Frontal Lobe, Parietal Lobe, Occipital Lobe, Temporal Lobe, Cingulate, and Basal Ganglia.

In various embodiments, the set of images or the second set of images comprise a stack of 2D images or a 2D representation of 3D images. In various embodiments, the first set of brain images and second set of brain images are magnetic resonance imaging (MRI) images. In various embodiments, the set of images and the second set of images captured from the individual comprise images of the individual's brain captured at two separate timepoints. In various embodiments, the set of images further comprise a set of combination images. In various embodiments, the set of images further comprise brain segmentation images comprising values that correlate locations within the brain segmentation to different brain regions. In various embodiments, the set of images further comprise a pre-existing lesion mask which includes values that categorize lesions into lesion types according to a location in the pre-existing lesion mask in which the lesion appears. In various embodiments, the anatomical abnormality is a lesion. In various embodiments, the characterization of the anatomical abnormality is a measure of multiple sclerosis (MS) disease activity or MS disease progression.

In various embodiments, the measure of MS disease activity is any one of: inter or intralesion relationships, lesion adjacency to neuroanatomy, intralesion voids (e.g., as a measure of permanent tissue damage or lesions within lesions), separated lesion surfaces from internal components, lesion characteristics (e.g., lesion surface, texture, shape, topology, density, homogeneity), temporal changes to lesions (e.g., new lesion, enlarging lesion, or shrinking lesion), and lesion volumetrics (e.g., total lesion load, merging, or splitting lesions).

In various embodiments, methods disclosed herein further comprise: displaying the representation of the 3D volume of the anatomical abnormality and the second representation of the 3D volume of the anatomical abnormality. In various embodiments, the displaying further comprises: displaying the characterization of the anatomical abnormality by displaying a transition from the representation of the 3D volume of the anatomical abnormality to the second representation of the 3D volume of the anatomical abnormality. In various embodiments, methods disclosed herein further comprise: based on the characterization of the anatomical abnormality, performing one or more of: performing a differential diagnosis of the individual's MS; selecting a candidate therapy for the individual; and determining an efficacy of a therapy previously administered to the individual.

In various embodiments, the 3D graph further comprises edges connecting the plurality of nodes. In various embodiments, one or more nodes of the plurality of nodes represent voxels in the 3D graph. In various embodiments, the one or more nodes are encoded with one or more of signal intensity information, spatial information, neighbor node information, temporal information, and anatomical information. In various embodiments, spatial information for a node comprises voxel coordinates of the node. In various embodiments, voxel coordinates comprise x, y, and z coordinates in the 3D graph for the node. In various embodiments, the signal intensity information comprises a signal intensity value. In various embodiments, the signal intensity value corresponds to a voxel in a combination image.

In various embodiments, temporal information comprises temporal features describing the node across two or more timepoints In various embodiments, adjacent nodes are defined by spatial characteristics relative to the seed node or relative to nodes that have been included in the node neighborhood during the iterative interrogation.

Additionally disclosed herein is a non-transitory computer readable medium comprising instructions that, when executed by a processor, cause the processor to: obtain a set of images captured from an individual, the set of images comprising an anatomical abnormality; generate a three dimensional (3D) graph using the set of images, the 3D graph comprising a plurality of nodes representing voxels and the anatomical abnormality; establish a seed node in the 3D graph indicative of a presence of the anatomical abnormality, the seed node defined by an initial voxel coordinate; define a node neighborhood comprising the seed node indicative of a 3D volume of the anatomical abnormality by: iteratively interrogate one or more adjacent nodes for inclusion or exclusion from the node neighborhood, wherein the interrogation of each of the one or more adjacent nodes is based on an intensity value of the adjacent node and an anatomical location of the adjacent node; generate a representation of the 3D volume of the anatomical abnormality; and store at least the representation of the 3D volume of the anatomical abnormality.

In various embodiments, the non-transitory computer readable medium further comprises instructions that, when executed by the processor, cause the processor to: obtain a second set of images captured from the individual, the second set of images further comprising the anatomical abnormality; generate a second three dimensional (3D) graph using the second set of images, the 3D graph comprising a plurality of nodes representing voxels and the anatomical abnormality; establish a seed node in the second 3D graph indicative of a presence of the anatomical abnormality in the second 3D graph, the seed node defined by an initial voxel coordinate; define a node neighborhood comprising the seed node indicative of a 3D volume of the anatomical abnormality by: iteratively interrogate one or more adjacent nodes for inclusion or exclusion from the node neighborhood, wherein the interrogation of each of the one or more adjacent nodes is based on an intensity value of the adjacent node and an anatomical location of the adjacent node; generate a second representation of the 3D volume of the anatomical abnormality; retrieve at least the stored representation of the 3D volume of the anatomical abnormality; characterize the anatomical abnormality by comparing the stored representation of the 3D volume of the anatomical abnormality to the second representation of the 3D volume of the anatomical abnormality.

In various embodiments, the instructions that cause the processor to interrogate the one or more adjacent nodes of the 3D graph or of the second 3D graph further comprise instructions that, when executed by the processor, cause the processor to: retrieve a threshold value previously determined for the anatomical location; and compare the intensity value of the adjacent node to the retrieved threshold value. In various embodiments, the non-transitory computer readable medium further comprises instructions that when executed by the processor, cause the processor to: responsive to the determination that the intensity value of the adjacent node exceeds the retrieved threshold value, include the adjacent node in the node neighborhood. In various embodiments, the non-transitory computer readable medium further comprises instructions that when executed by the processor, cause the processor to: responsive to the determination that the intensity value of the adjacent node is less than the retrieved threshold value, exclude the adjacent node from the node neighborhood.

In various embodiments, the instructions that cause the processor to interrogate of the one or more adjacent nodes of the 3D graph or of the second 3D graph further comprise instructions that, when executed by the processor, cause the processor to: determine whether the anatomical location of the adjacent pixel differs from an anatomical location of the seed node. In various embodiments, the non-transitory computer readable medium further comprises instructions that, when executed by the processor, cause the processor to: responsive to the determination that the anatomical location of the adjacent node does not differ from the anatomical location of the seed node, include the adjacent node in the node neighborhood. In various embodiments, the non-transitory computer readable medium further comprises instructions that, when executed by the processor, cause the processor to: responsive to the determination that the anatomical location of the adjacent node differs from the anatomical location of the seed node, exclude the adjacent node in the node neighborhood. In various embodiments, the anatomical location of the node is a neuroanatomical location of the node. In various embodiments, the neuroanatomical location of the node comprises one or more of 3rd Ventricle, 4th Ventricle, 5th Ventricle, Amygdala, Anterior Cingulate, Anterior Middle Frontal, Brainstem, Caudal Anterior Cingulate, Caudate, Cerebellar Gray Matter, Cerebellar White Matter, Cerebral White Matter, Cerebral WM Hypointensities, Cortical Gray Matter, Cuneus, Entorhinal Cortex, Frontal Pole, Fusiform, Hippocampus, Inferior Frontal, Inferior Lateral Ventricles, Inferior Parietal, Inferior Temporal, Insula, Isthmus Cingulate, Lateral Occipital, Lateral Orbitofrontal, Lingual, Medial Occipital, Medial Orbitofrontal, Medial Parietal, Middle Frontal, Middle Temporal, Nucleus Accumbens, Pallidum, Paracentral, Parahippocampal, Pars Opercularis, Pars Orbitalis, Pars Triangularis, Pericalcarine, Posterior Cingulate, Posterior Superior Temporal Sulcus, Premotor, Primary Motor, Primary Sensory, Putamen, Rostral Anterior Cingulate, Superior Frontal, Superior Lateral Ventricles, Superior Parietal, Superior Temporal, Supramarginal, Temporal Pole, Thalamus, Transverse Temporal, Transverse Temporal+Superior Temporal, Ventral Diencephalon, Whole Brain, Intracranial Volume, Forebrain Parenchyma, Ventricles, Cerebellum, Frontal Lobe, Parietal Lobe, Occipital Lobe, Temporal Lobe, Cingulate, and Basal Ganglia.

In various embodiments, the set of images or the second set of images comprise a stack of 2D images or a 2D representation of 3D images. In various embodiments, the first set of brain images and second set of brain images are magnetic resonance imaging (MRI) images. In various embodiments, the set of images and the second set of images captured from the individual comprise images of the individual's brain captured at two separate timepoints. In various embodiments, the set of images further comprise a set of combination images. In various embodiments, the set of images further comprise brain segmentation images comprising values that correlate locations within the brain segmentation to different brain regions. In various embodiments, the set of images further comprise a pre-existing lesion mask which includes values that categorize lesions into lesion types according to a location in the pre-existing lesion mask in which the lesion appears In various embodiments, the anatomical abnormality is a lesion.

In various embodiments, the characterization of the anatomical abnormality is a measure of multiple sclerosis (MS) disease activity or MS disease progression. In various embodiments, the measure of MS disease activity is any one of: inter or intralesion relationships, lesion adjacency to neuroanatomy, intralesion voids (e.g., as a measure of permanent tissue damage or lesions within lesions), separated lesion surfaces from internal components, lesion characteristics (e.g., lesion surface, texture, shape, topology, density, homogeneity), temporal changes to lesions (e.g., new lesion, enlarging lesion, or shrinking lesion), and lesion volumetrics (e.g., total lesion load, merging, or splitting lesions).

In various embodiments, the non-transitory computer readable medium further comprises instructions that, when executed by the processor, cause the processor to: display the representation of the 3D volume of the anatomical abnormality and the second representation of the 3D volume of the anatomical abnormality. In various embodiments, the instructions that cause the processor to display further comprise instructions that, when executed by the processor, cause the processor to: display the characterization of the anatomical abnormality by displaying a transition from the representation of the 3D volume of the anatomical abnormality to the second representation of the 3D volume of the anatomical abnormality.

In various embodiments, the non-transitory computer readable medium further comprises instructions that, when executed by the processor, cause the processor to: based on the characterization of the anatomical abnormality, perform one or more of: perform a differential diagnosis of the individual's MS; select a candidate therapy for the individual; and determine an efficacy of a therapy previously administered to the individual.

In various embodiments, the 3D graph further comprises edges connecting the plurality of nodes. In various embodiments, one or more nodes of the plurality of nodes representing voxels in the 3D graph. In various embodiments, the one or more nodes are encoded with one or more of signal intensity information, spatial information, neighbor node information, temporal information, and anatomical information. In various embodiments, spatial information for a node comprises voxel coordinates of the node. In various embodiments, voxel coordinates comprise x, y, and z coordinates in the 3D graph for the node. In various embodiments, the signal intensity information comprises a signal intensity value. In various embodiments, the signal intensity value corresponds to a voxel in a combination image. In various embodiments, the temporal information comprises temporal features describing the node across two or more timepoints. In various embodiments, adjacent nodes are defined by spatial characteristics relative to the seed node or relative to nodes that have been included in the node neighborhood during the iterative interrogation.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “node 220A,” indicates that the text refers specifically to the element having that particular reference numeral. A reference numeral in the text without a following letter, such as “node 220,” refers to any or all of the elements in the figures bearing that reference numeral (e.g. “node 220” in the text refers to reference numerals “node 220A” and/or “node 220B” in the figures).

Figure (FIG. 1A depicts a system environment overview implementing 3D graphs, in accordance with an embodiment.

FIG. 1B depicts a block diagram of the graph system, in accordance with an embodiment.

FIG. 2A depicts an example encoding of a set of images into a 3D graph, in accordance with an embodiment.

FIG. 2B depicts example nodes of a 3D graph, in accordance with an embodiment.

FIG. 3A depicts a first step of determining a node neighborhood involving the identification of a seed node, in accordance with an embodiment.

FIG. 3B depicts a second step of determining a node neighborhood involving the interrogation of adjacent nodes, in accordance with an embodiment.

FIG. 3C depicts an example node neighborhood indicative of an anatomical abnormality, in accordance with the embodiments shown in FIGS. 3A and 3B.

FIG. 4 is a flow process for generating a representation of an anatomical abnormality in a 3D graph, in accordance with an embodiment.

FIG. 5A depicts the implementation of an updated three dimensional graph for determining a temporal change of the anatomical abnormality, in accordance with an embodiment.

FIG. 5B depicts the interrogation of additional nodes in the updated three dimensional graph for determining a temporal change of the anatomical abnormality, in accordance with an embodiment.

FIG. 5C depicts an example updated node neighborhood indicative of an anatomical abnormality, in accordance with the embodiments shown in FIGS. 5A and 5B.

FIG. 6 depicts an example transition between the node neighborhood and updated node neighborhood, in accordance with an embodiment.

FIG. 7 illustrates an example computer for implementing the entities shown in FIGS. 1A and 1B.

FIG. 8A depicts an example 3D graph with individual nodes that are connected to other nodes through edges (e.g., connections).

FIG. 8B shows characterization and quantification of nodes within node neighborhoods defining lesions.

FIG. 8C and FIG. 8D each show the identification of a lesion within the brain using different minimum threshold values.

FIG. 8E depicts an example lesion community, lesion surface, and lesion shell that are defined using a 3D graph.

FIGS. 9A and 9B depicts the growing and merging of lesion bodies using a 3D graph.

FIG. 10A depicts a lesion splitting within a 3D graph.

FIG. 10B depicts a lesion splitting and merging within a 3D graph.

FIG. 10C depicts a shrinking lesion within a 3D graph.

FIG. 10D depicts a changing shape of a lesion within a 3D graph.

### DETAILED DESCRIPTION

### I. Definitions

Terms used in the claims and specification are defined as set forth below unless otherwise specified.

The terms “subject” or “patient” are used interchangeably and encompass a cell, tissue, or organism, human or non-human, male, or female.

The term “obtaining one or more images” encompasses obtaining one or more images captured from a subject. Obtaining one or more images can encompass performing steps of capturing the one or more images e.g., using an imaging device. The phrase can also encompass receiving one or more images, e.g., from a third party that has performed the steps of capturing the one or more images from the subject. The one or more images can be obtained by one of skill in the art via a variety of known ways including stored on a storage memory. The term “obtaining one or more images” can also include having (e.g., instructing) a 3rd party obtain the one or more images.

The phrase “3D graph” refers to a three dimensional graph composed of a plurality of nodes and edges. As described herein, a 3D graph is useful for identifying anatomical abnormalities and characterizing disease activity e.g., multiple sclerosis disease activity.

The term “node’ refers to an element of the 3D graph. In various embodiments, each node corresponds to a voxel within the 3D graph. Each node can further be encoded with additional information such as any of signal intensity information, spatial information, neighbor node information, temporal information, and anatomical information.

The terms “connection” and “edge” are used interchangeably and represent linkages between nodes within a 3D graph. In various embodiments, nodes that are adjacent to one another are connected via a connection or edge within the 3D graph.

The phrase “node neighborhood” refers to one or more nodes within the 3D graph that are indicative of an anatomical abnormality. In various embodiments, a node neighborhood is identified through an iterative interrogation process of the nodes of the 3D graph.

The terms “treating,” “treatment,” or “therapy” shall mean slowing, stopping or reversing a progression of a disease by administration of treatment. In some embodiments, treating a disease means reversing the disease's progression, ideally to the point of eliminating the disease itself. In various embodiments, “treating,” “treatment,” or “therapy” includes administering a therapeutic agent or pharmaceutical composition to the subject.

The phrase “administering a therapeutic agent” or “administering a composition” includes providing, to a subject, a therapeutic agent or pharmaceutical composition. In various embodiments, the therapeutic agent or composition can be provided for prophylactic purposes. Prophylaxis of a disease refers to the administration of a composition or therapeutic agent to prevent the occurrence, development, onset, progression, or recurrence of a disease or some or all of the symptoms of the disease or to lessen the likelihood of the onset of the disease.

It must be noted that, as used in the specification, the singular forms “a,” “an” and “the” include plural referents unless the context clearly dictates otherwise.

### II. System Environment Overview

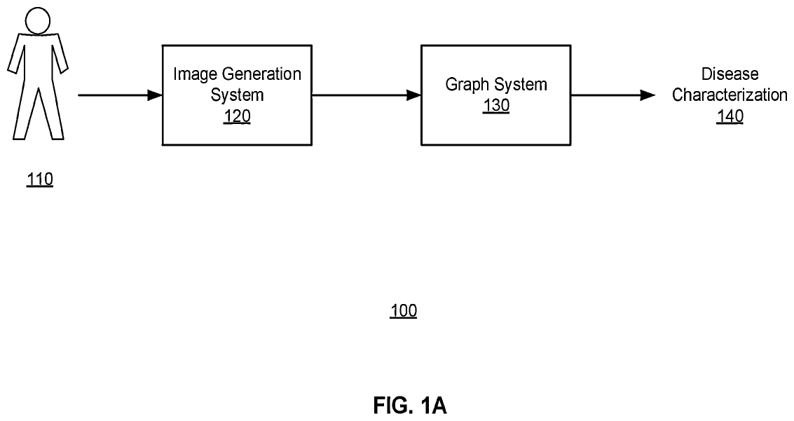

Figure (FIG. 1A depicts a system environment overview implementing 3D graphs, in accordance with an embodiment. The system environment 100 provides context in order to introduce a subject 110, an image generation system 120, and a graph system 130 for determining a disease characterization 140 for the subject 110. Although FIG. 1A depicts one subject 110 for whom a disease characterization 140 is generated, in various embodiments, the system environment 100 includes two or more subjects such that that graph system 130 generates disease characterizations 140 for the two or more subjects (e.g., a disease characterization for each of the two or more subjects). In various embodiments, a disease characterization can be useful for guiding treatment for the subject 110. For example, the disease characterization 140 can indicate topological features and/or temporal changes of the disease, which can be used to guide whether a subject 110 is to be provided an intervention.

In various embodiments, the subject was previously diagnosed with a disease. Thus, the disease characterization 140 for the subject can be useful for determining a presence or absence of the disease. In various embodiments, the subject is suspected of having a disease. Therefore, the disease characterization 140 for the subject shown in FIG. 1A can be useful for diagnosing the patient with the disease. In particular embodiments, the disease is a neurodegenerative disease, such as multiple sclerosis. In particular embodiments, the disease is a cancer. Additional examples of diseases are described herein.

Referring to FIG. 1A, the image generation system 120 captures one or more images from the subject 110. In various embodiments, the image can be obtained by a third party, e.g., a medical professional. Examples of medical professionals include physicians, emergency medical technicians, nurses, first responders, psychologists, phlebotomist, medical physics personnel, nurse practitioners, surgeons, dentists, and any other obvious medical professional as would be known to one skilled in the art. In various embodiments, the image can be obtained in a hospital setting or a medical clinic.

In various embodiments, the image generation system 120 captures one or more images of the full body of the subject 110. In various embodiments, the image generation system 120 captures one or more images from a particular anatomical location of the subject 110. For example, the image generation system 120 may capture one or more images from an anatomical organ of the subject. In various embodiments, the image generation system 120 performs a scan across the full anatomical organ, thereby capturing one or more images of the full anatomical organ. Example organs include the brain, heart, thorax, lung, abdomen, colon, cervix, pancreas, kidney, liver, muscle, lymph nodes, esophagus, intestine, spleen, stomach, and gall bladder. In particular embodiments, the image generation system 120 captures one or more images of the subject's brain.

In various embodiments, the image generation system 120 captures various sets of one or more images of the subject 110. For example, the image generation system 120 may capture a first set of images of the subject 110 prior to administering an agent. The image generation system 120 may further capture a second set of images of the subject 110 after administering the agent. Examples of an agent include a contrast agent, such as a MRI contrast agent (e.g., gadolinium). Therefore, the first set of images and the second set of images can represent pre-contrast and post-contrast images, respectively, captured from the subject 110.

In various embodiments, the imaging generation system 120 includes an imaging device for capturing the one or more images. The imaging device can be one of a computed tomography (CT) scanner, magnetic resonance imaging (MRI) scanner, positron emission tomography (PET) scanner, x-ray scanner, an ultrasound imaging device, or a light microscope, such as any of a brightfield microscope, darkfield microscope, phase-contrast microscope, differential interference contrast microscope, fluorescence microscope, confocal microscope, or two-photon microscope. In particular embodiments, the imaging device is a MRI scanner that captures MRI images. In particular embodiments, the imaging device is a MRI scanner that captures a set of two dimensional (2D) images, such as a 2D stack of MRI images.

Generally, the graph system 130 generates a three dimensional (3D) graph using the one or more images captured from the subject 110 (e.g., images captured by the imaging generation system 120) and uses the 3D graph to generate the disease characterization 140 for the subject 110. In various embodiments, the disease characterization 140 is an indication of topological features and/or temporal changes of the disease. For example, the disease characterization 140 can be an indication that an anatomical abnormality associated with the disease is present, and therefore, the subject has the disease. As another example, the disease characterization 140 can be an indication that an anatomical abnormality associated with the disease is changing (e.g., increasing in size, decreasing in size, or changing shape) and therefore, the disease is progressing or reverting.

In various embodiments, the disease characterization 140 can include a treatment recommendation for the subject 110 based on the topological and/or temporal changes of the disease. In one scenario, the subject 110 may be receiving an intervention. If the graph system 130 uses the 3D graph and determines that the subject 110 is experiencing disease progression, the disease characterization 140 can include a treatment recommendation that suggests a different therapeutic intervention. In contrast, if the subject 110 is receiving an intervention and the graph system 130 determines that the subject 110 is experiencing disease reversion, the disease characterization 140 can include a treatment recommendation that suggests continuation of the current intervention.

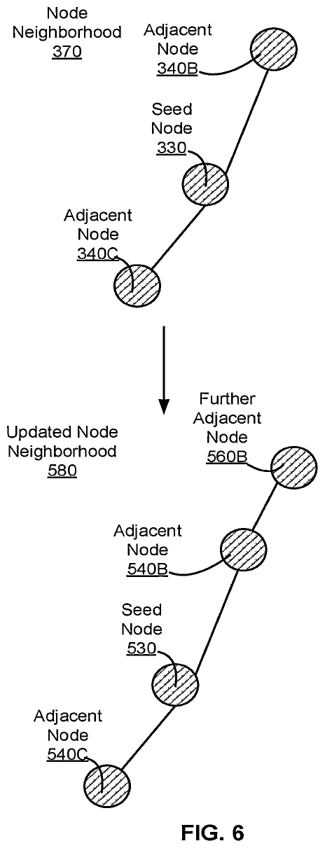

The graph system 130 can include one or more computers, embodied as a computer system 700 as discussed below with respect to FIG. 7. Therefore, in various embodiments, the steps described in reference to the graph system 130 are performed in silico. In various embodiments, the imaging generation system 120 and the graph system 130 are employed by different parties. For example, a first party operates the imaging generation system 120 to capture one or more images derived from the subject 110 and then provides the captured one or more images to a second party which implements the graph system 130 to determine a disease characterization 140. In some embodiments, the imaging generation system 120 and the graph system 130 are employed by the same party.

Reference is now made to FIG. 1B, which depicts a block diagram of the graph system 130, in accordance with an embodiment. Here, the graph system 130 includes a graph encoding module 145, an abnormality identifier module 150, a disease characterization module 160, and a graph store 170. In various embodiments, the graph system 130 can be configured differently with additional or fewer modules.

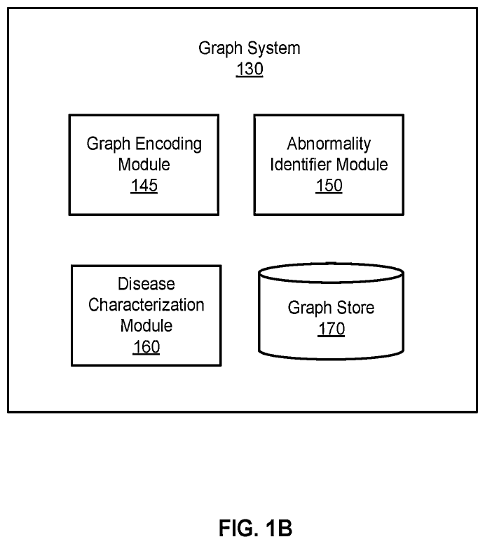

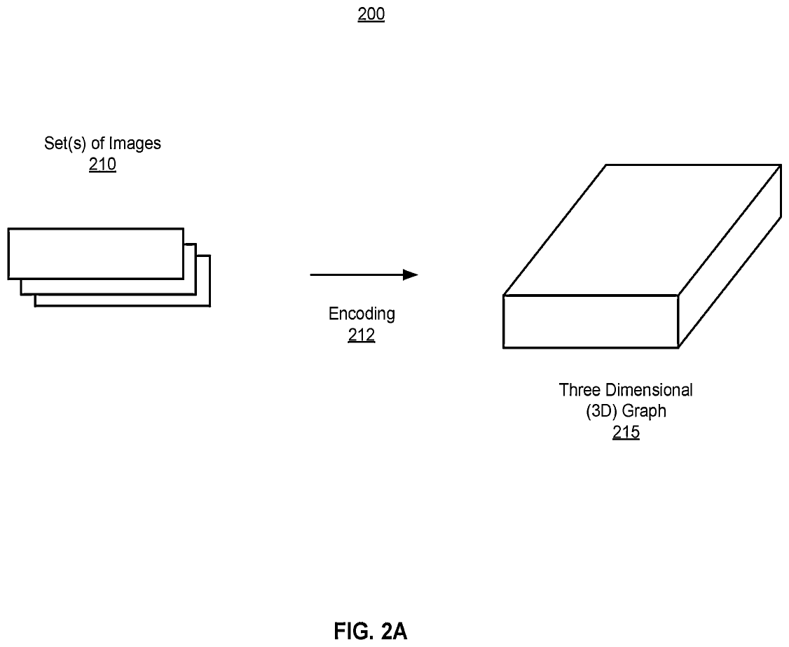

Referring to the graph encoding module 145, it encodes one or more images (e.g., images captured by the imaging generation system 120) into a three dimensional (3D) graph structure. In various embodiments, the one or more images represent a stack of two dimensional (2D) images and therefore, the graph encoding module 145 can encode the stack of 2D images into the 3D graph structure. In particular embodiments, the one or more images are a stack of MRI images captured from the subject's brain. Thus, the graph encoding module 145 encodes the stack of MRI images into a 3D graph structure of the subject's brain. Generally, the 3D graph structure includes a plurality of nodes in which nodes are connected to other nodes through connections. In various embodiments, each node represents a voxel that defines the spatial location of the node within the 3D graph structure. In various embodiments, the graph encoding module 145 encodes additional information within each nodule, examples of which include signal intensity information, spatial information, neighbor node information, temporal information, and anatomical information. The 3D graph is described in further detail below in reference to FIG. 2B.

Referring next to the abnormality identifier module 150, it analyzes the nodes of the 3D graph to identify one or more anatomical abnormalities within the 3D graph. For an anatomical abnormality, the abnormality identifier module 150 generates a node neighborhood including one or more nodes that is representative of the anatomical abnormality. Nodes included in the node neighborhood indicate presence of the anatomical abnormality at the location of the node within the 3D graph. The abnormality identifier module 150 identifies a seed node that is indicative of an anatomical abnormality and performs an iterative process to interrogate nodes that are adjacent to the seed node to determine whether to include or exclude each adjacent node within the node neighborhood. Thus, through this iterative process, the abnormality identifier module 150 generates a node neighborhood within the 3D graph that is representative of the anatomical abnormality. The abnormality identifier module 150 can store the node neighborhoods that represent anatomical abnormalities into the graph store 170.

In various embodiments, the abnormality identifier module 150 identifies anatomical abnormalities within 3D graphs that correspond to different timepoints. For example, the abnormality identifier module 150 identifies an anatomical abnormality within a 3D graph that is generated from a set of images captured from a subject at a first timepoint. Furthermore, the abnormality identifier module 150 identifies the anatomical abnormality within a 3D graph that is generated from a set of images captured from the subject at a second timepoint. Thus, the difference between the anatomical abnormality at the different timepoints represents the change in the anatomical abnormality across the different timepoints.

Referring next to the disease characterization module 160, it analyzes the anatomical abnormalities identified by the abnormality identifier module 150 and generates a disease characterization (e.g., disease characterization 140 as described in reference to FIG. 1A). In various embodiments, the disease characterization module 160 determines a disease characterization based on an analysis of an anatomical abnormality from a single timepoint. For example, the disease characterization module 160 may determine a disease characterization based on single timepoint characteristics of the anatomical abnormality, including inter or intra-abnormality relationships, abnormality adjacency to anatomical landmarks, intra-abnormality voids (e.g., as a measure of tissue damage within an abnormality), separated abnormality surfaces from internal components, abnormality characteristics (e.g., surface, texture, shape, topology, density, homogeneity), abnormality volumetrics (e.g., total abnormality load). In various embodiments, the disease characterization module 160 determines a disease characterization based on an analysis of an anatomical abnormality from two different timepoints. Thus, the disease characterization module 160 further considers the change in the anatomical abnormality across two or more timepoints. The changes in the anatomical abnormality can include a change in inter or intra-abnormality relationships, change in abnormality adjacency to anatomical landmarks, change in intra-abnormality voids (e.g., as a measure of tissue damage within an abnormality), change in separated abnormality surfaces from internal components, change in abnormality characteristics (e.g., change in any of surface, texture, shape, topology, density, homogeneity), change in abnormality volumetrics (e.g., change in total abnormality load, merging or splitting abnormalities).

### III. Three Dimensional Graph

FIG. 2A depicts an example encoding of one or more sets of images into a 3D graph, in accordance with an embodiment. Generally, the steps described here in reference to FIG. 2A can be performed by the graph encoding module 145 described above in reference to FIG. 1B.

Referring to the one or more sets of images 210, they include at least images captured from the subject (e.g., images captured by the image generation system 120). In various embodiments, the images captured from the subject include computed tomography (CT) images, such as a 2D stack of CT images. In various embodiments, the images captured from the subject include MRI images, such as a 2D stack of MRI images. In particular embodiments, the images from the subject include images (e.g., MRI or CT images) of the subject's brain. Examples of MRI images include one or both of T1 weighted images, T2 weighted images, or fluid attenuated inversion recovery (FLAIR) images. In particular embodiments, the one or more sets of images 210 include T1 weighted images. In particular embodiments, the one or more sets of images 210 include FLAIR images. In particular embodiments, the one or more sets of images 210 include a set of T1 weighted images and a set of FLAIR images. In particular embodiments, the one or more sets of images 210 include a set of T1-weighted FLAIR images.

In various embodiments, the one or more sets of images 210 includes combination images. Generally, combination images represent a combination between different image acquisitions. For example, combination images can be any one of multiplication images, division images, or subtraction images.

Multiplication images represent the calculated multiplication of values of different image acquisitions. For example, values of pixels or voxels of a first set of images can be multiple with values of pixels or voxels of a second set of images. Division images represent the calculated division of values of different image acquisitions. For example, values of pixels or voxels of a first set of images can be divided by values of pixels or voxels of a second set of images, or vice versa.

Subtraction images represent calculated differences between different image acquisitions. In various embodiments, different image acquisitions can refer to sets of images acquired through different imaging modalities. For example, different image acquisitions can refer to T1 v. T2 images. Therefore, subtraction images can refer to calculated differences between captured T1 images and captured T2 images. As another example, different image acquisitions can refer to different types of imaging, such as MRI v. CT imaging. Therefore, subtraction images can refer to calculated differences between captured MRI images and captured CT images.

In various embodiments, different image acquisitions can refer to sets of images acquired at different timepoints e.g., a first set of images acquired at a first timepoint and a second set of images acquired at a second timepoint. In various embodiments, the set of images acquired at a first timepoint represent pre-contrast images. In various embodiments, the set of images acquired at a second timepoint represent post-contrast images. Pre-contrast images can refer to images captured of a subject prior to administration of a contrast agent (e.g., a MRI contrast agent such as gadolinium). Post-contrast images can refer to images captured of a subject after administration of a contrast agent (e.g., a MRI contrast agent such as gadolinium). As one example, subtraction images may represent calculated differences between pre-contrast and post-contrast T1-weighted images. As another example, subtraction images may represent calculated differences between pre-contrast and post-contrast FLAIR images. As another example, subtraction images may represent calculated differences between pre-contrast and post-contrast T1-weighted FLAIR images. In various embodiments, subtraction images may represent calculated differences between normalized pre-contrast images and normalized post-contrast images. For example, the pre-contrast images and post-contrast images may be separately normalized via Z-score normalization.

In various embodiments, the one or more sets of images 210 further include previously generated images correlating locations within the images to different anatomical regions. For example, in particular embodiments, the previously generated images can correlate locations within the images to different brain regions. These images, hereafter referred to as brain segmentation images, are useful for segmenting the brain within the 3D graph into different brain regions. Example brain regions include, but are not limited to, 3rd Ventricle, 4th Ventricle, 5th Ventricle, Amygdala, Anterior Cingulate, Anterior Middle Frontal, Brainstem, Caudal Anterior Cingulate, Caudate, Cerebellar Gray Matter, Cerebellar White Matter, Cerebral White Matter, Cerebral WM Hypointensities, Cortical Gray Matter, Cuneus, Entorhinal Cortex, Frontal Pole, Fusiform, Hippocampus, Inferior Frontal, Inferior Lateral Ventricles, Inferior Parietal, Inferior Temporal, Insula, Isthmus Cingulate, Lateral Occipital, Lateral Orbitofrontal, Lingual, Medial Occipital, Medial Orbitofrontal, Medial Parietal, Middle Frontal, Middle Temporal, Nucleus Accumbens, Pallidum, Paracentral, Parahippocampal, Pars Opercularis, Pars Orbitalis, Pars Triangularis, Pericalcarine, Posterior Cingulate, Posterior Superior Temporal Sulcus, Premotor, Primary Motor, Primary Sensory, Putamen, Rostral Anterior Cingulate, Superior Frontal, Superior Lateral Ventricles, Superior Parietal, Superior Temporal, Supramarginal, Temporal Pole, Thalamus, Transverse Temporal, Transverse Temporal+Superior Temporal, Ventral Diencephalon, Whole Brain, Intracranial Volume, Forebrain Parenchyma, Ventricles, Cerebellum, Frontal Lobe, Parietal Lobe, Occipital Lobe, Temporal Lobe, Cingulate, and Basal Ganglia.

In various embodiments, the one or more sets of images 210 further include a pre-existing lesion mask which categorizes lesions into particular lesion types according to the location in which the lesion appears. As an example, a lesion mask is defined as an image where the intensities are discrete values that map to labels (for example but not limited to lesion types, brain anatomical regions). For example, the pre-existing lesion mask may be a stack of 2D images or a 3D image with values arranged in an array corresponding to lesion types. Example lesion types include juxtacortical, periventricular, deep white, or infratentorial lesion types.

In various embodiments, the one or more sets of images 210 further include blank images. These blank images can be useful for adding newly identified anatomical abnormalities.

In various embodiments, the one or more sets of images 210 include one or more of 1) MRI images captured from the subject, 2) combination images, 3) brain segmentation images, and 4) pre-existing lesion mask. In various embodiments, the one or more sets of images 210 include each of 1) MRI images captured from the subject, 2) combination images, 3) brain segmentation images, and 4) pre-existing lesion mask.

As shown in FIG. 2A, the one or more sets of images 210 are encoded 212 (e.g., encoded by the graph encoding module 145 described in FIG. 1B) to generate the three dimensional (3D) graph 215. Generally, the 3D graph 215 includes a plurality of nodes, in which nodes are connected to other nodes through connections. In various embodiments, each node represents a voxel that defines the spatial location of the node within the 3D graph. Thus, a particular node can be connected to adjacent nodes that are spatially located next to the particular node.

In various embodiments, the graph encoding module 145 can encode the information available in the one or more sets of images 210 into the nodes or edges (also referred to as connections) of the 3D graph 215. For example, the graph encoding module 145 can encode one or more of signal intensity information, spatial information, neighbor node information, temporal information, and anatomical information into each of the nodes and/or into edges of the 3D graph 215.

In various embodiments, signal intensity information encoded in a node includes signal intensity of the corresponding voxel from the MRI images captured from the subject. In various embodiments, signal intensity information encoded in a node includes signal intensity of a corresponding voxel in the combination image. In various embodiments, spatial information can include an identification of the spatial location of the node within the 3D graph. For example, spatial information can include the coordinates (e.g., x, y, and z coordinates) of the node within the 3D graph.

In various embodiments, neighbor node information for a node includes information identifying the one or more adjacent nodes that the node is connected to. For example, neighbor node information can identify whether an adjacent node is a neighbor in any one of the x coordinate, they coordinate, or the z coordinate. As another example, neighbor node information can identify whether an adjacent is node is a diagonal neighbor or bisect. In various embodiments, neighbor node information may further identify whether an adjacent node is in the same anatomical location as the node or in a different anatomical location as the node. In various embodiments, the neighbor node information for a node is encoded within the node. In various embodiments, the neighbor node information for a node is encoded within an edge connecting a node and an adjacent node. For example, given that the neighbor node information describes the neighboring relationship between a node and an adjacent node, the neighbor node information can be encoded within a connection between the node and the adjacent node.

In various embodiments, temporal information for a node refers to information corresponding to the node for one or more timepoints. For example, temporal information for a node can identify when a first set of MRI images were captured and used to build the 3D graph. Temporal information for the node can further identify when subsequent sets of MRI images were captured and used to build the 3D graph. In various embodiments, anatomical information encoded in a node refers to a value indicating the brain region that the node is located in. Anatomical information can be derived from the brain segmentation images.

In various embodiments, a node in the 3D graph includes at least one adjacent node. Generally, an adjacent node of a particular node is spatially located next to the particular node. For example, if the coordinates of the particular node is (a, b, c), then the coordinates of an adjacent node can be 1 unit away in any of the x, y, or z directions (e.g., coordinates of (a±1, b, c), (a, b±1, c), or (a, b, c±1)).

In various embodiments, a node in the 3D graph includes at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, at least twenty one, at least twenty two, at least twenty three, at least twenty four, or at least twenty five adjacent nodes. In particular embodiments, a node in the 3D graph includes twenty six adjacent nodes.

Reference is now made to FIG. 2B, which depicts example nodes of a 3D graph 215, in accordance with an embodiment. One skilled in the art may appreciate that the 3D graph shown in FIG. 2B is merely exemplary, and in other embodiments, there may be tens, hundreds, thousands, tens of thousands, hundreds of thousands, or millions of nodes in a 3D graph 215. Here, FIG. 2B shows nodes 220A, 220B, 220C, 220D, 220E, 220F, 220G, and 220H. As described above, each node can be encoded with information, such as one or more of signal intensity information, spatial information, neighbor node information, temporal information, and anatomical information.

As shown in FIG. 2B, nodes are linked to other nodes in the 3D graph 215 through connections (e.g., connection 225A and connection 228A). Generally, a first node that is linked to second node is referred to as an adjacent node of the second node. Thus, in FIG. 2B, node 220A is an adjacent node to each of node 220B, node 220C, and node 220F. Additionally, node 220B is an adjacent node to each of node 220A, node 220C, and node 220D.

FIG. 2B further shows that nodes 220A, 220B, 220C, 220D, and 220E are located within the 3D graph 215 in a first anatomical location 230A (e.g., anatomical location as determined based on anatomical information derived from brain segmentation images). Additionally, nodes 220F, 220G, and 220H are located within the 3D graph 215 in a second anatomical location 230B. For example, anatomical location 230A may be white matter in the brain whereas anatomical location 230B may be grey matter in the brain.

In various embodiments, the connections between adjacent nodes may differ depending on whether the adjacent nodes are in the same anatomical location or in different anatomical locations. In FIG. 2B, the different connections are indicated by the solid line connections (e.g., connection 225A) and the dotted line connection (e.g., connection 228A). As way of example, node 220A and node 220B are adjacent nodes and are linked through connection 225A as they are both in the same anatomical location 230A. In contrast, node 220A and node 220F are adjacent nodes and are linked through a different connection 228A as they are in different anatomical locations. Given that the brain includes various anatomical regions, by differently linking adjacent nodes based on their same or different anatomical locations can be useful e.g., useful for identifying an anatomical abnormality in the 3D graph as discussed below.

### IV. Example Process for Identifying an Anatomical Abnormality Within a 3D Graph

Generally, the steps described here for identifying an anatomical abnormality (e.g., a lesion) can be performed by the abnormality identifier module 150 described above in reference to FIG. 1B. The process for identifying an anatomical abnormality in the 3D graph involves determining a node neighborhood including one or more nodes that are indicative of an anatomical abnormality. In various embodiments, the process involves first identifying a seed node in the 3D graph for inclusion in the node neighborhood, the seed node likely indicative of the anatomical abnormality. Additional nodes are next interrogated to determine whether the additional nodes are to be included or excluded from the node neighborhood. In various embodiments, the inclusion or exclusion of additional nodes involves an iterative process. For example, adjacent nodes of the seed node (e.g., nodes connected to the seed node) are interrogated to determine whether to include or exclude the adjacent nodes in the node neighborhood. Next, for each adjacent node that is included in the node neighborhood, additional nodes that are connected to the adjacent node are interrogated to determine whether the additional nodes are to be included or excluded from the node neighborhood. The end result of this process is a node neighborhood including a plurality of nodes that have been interrogated and determined to be likely indicative of the anatomical abnormality. Thus, the node neighborhood within the 3D graph represents the anatomical abnormality.

In various embodiments, a seed node in a node neighborhood is identified using a label that identifies the seed node as being located within an anatomical abnormality. For example, the label can be derived based on user input (e.g., a user can select a node to be a seed node). In various embodiments, a seed node in a node neighborhood is identified by querying the information encoded within the seed node. As one example, a node can be identified as a seed node if the signal intensity of the corresponding voxel is above a threshold value. In various embodiments, the threshold value is set according to a statistical measure of the signal intensities of nodes. In one embodiment, the threshold value may be X % of a max signal intensity across all nodes in the 3D graph. In one embodiment, the threshold value may be X % of a max signal intensity across each anatomical region (e.g., each brain region) in the 3D graph. In various embodiments, X is 90%, 91%, 92%, 93%, 94%, 95%, 96%, 97%, 98%, 99%, or 100%. In various embodiments, all nodes in the 3D graph or all nodes in an anatomical region with a signal intensity value above X % of the max signal intensity can be selected for inclusion in a node neighborhood. In various embodiments, all nodes in the 3D graph or all nodes in an anatomical region with a signal intensity value above X % of the max signal intensity can be selected as seed nodes. In various embodiments, the threshold value is set such that the top Y nodes in the 3D graph with the highest signal intensity are selected as seed nodes. In various embodiments, the threshold value is set such that the top Y % nodes in each anatomical region (e.g., each brain anatomical region) with the highest signal intensity are selected as seed nodes. For example, Y can be 0.5%, 1%, 2%, 3%, 4%, 5%, 6%, 7%, 8%, 9%, or 10%. Therefore, Y % of all nodes in the 3D graph or Y % of all nodes in an anatomical region can be selected as a seed node. In various embodiments, after a seed node is selected, a seed node can be further unlabeled. For example, a seed node can be unlabeled based on user input (e.g., a user can de-select a node as a seed node if a seed node is mistakenly identified).

In various embodiments, the interrogation of a node involves comparing different information encoded within the node to determine whether the node is to be included or excluded from the node neighborhood. In particular embodiments, the interrogation of a node involves comparing signal intensity information of the node.

As a first example of comparing signal intensity information of the node, the interrogation of a node involves comparing a signal intensity of the corresponding voxel from images captured from the subject (e.g., post-contrast MRI image captured from the subject) to a signal intensity of a corresponding voxel of the combination images. If the signal intensity of the corresponding voxel from images is greater than the signal intensity of a corresponding voxel of the combination images, the node is included in the node neighborhood. If the signal intensity of the corresponding voxel from images is less than the signal intensity of a corresponding voxel of the combination images, the node is excluded from the node neighborhood.

As another example of comparing signal intensity information of the node, the interrogation of a node involves establishing a minimum threshold and comparing the signal intensity information of the node to the established minimum threshold. In various embodiments, the minimum threshold is established as the signal intensity of a voxel in the combination images that corresponds to the seed node. Thus, the minimum threshold can be a fixed value for comparing signal intensity information of each of the subsequent adjacent nodes. Thus, when interrogating a node, if the signal intensity of the corresponding voxel from images (e.g., post-contrast MRI images) is greater than the minimum threshold, the node is included in the node neighborhood. If the signal intensity of the corresponding voxel from images (e.g., post-contrast MRI images) is less than the minimum threshold, the node is excluded from the node neighborhood. In various embodiments, the minimum threshold can be any one of −1.0, −0.9, −0.8, −0.7, −0.6, −0.5, −0.4, −0.3, −0.2, −0.1, 0, 0.1, 0.2, 0.3, 0.4, 0.5, 0.6, 0.7, 0.8, 0.9, or 1.0. In particular embodiments, the minimum threshold is 0.5. In particular embodiments, the minimum threshold is −0.4.

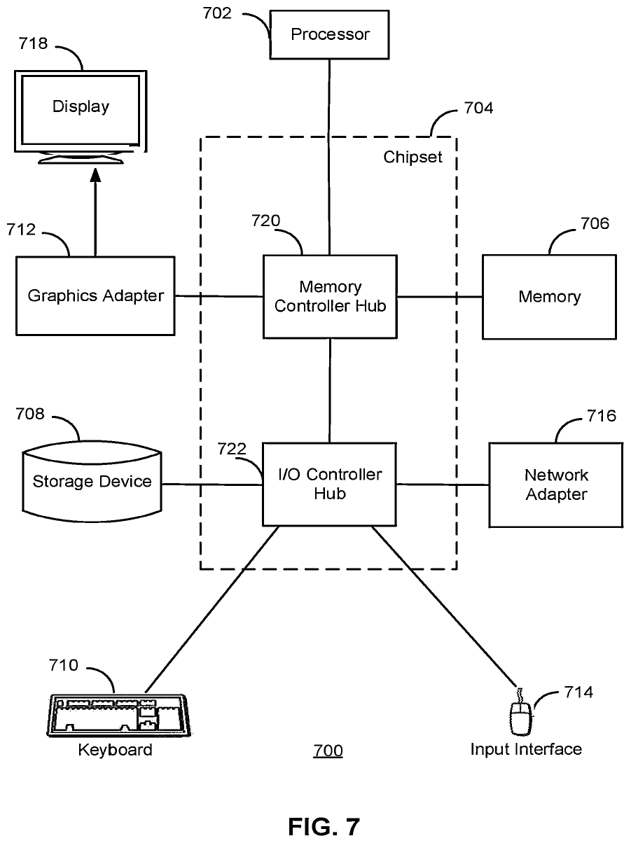

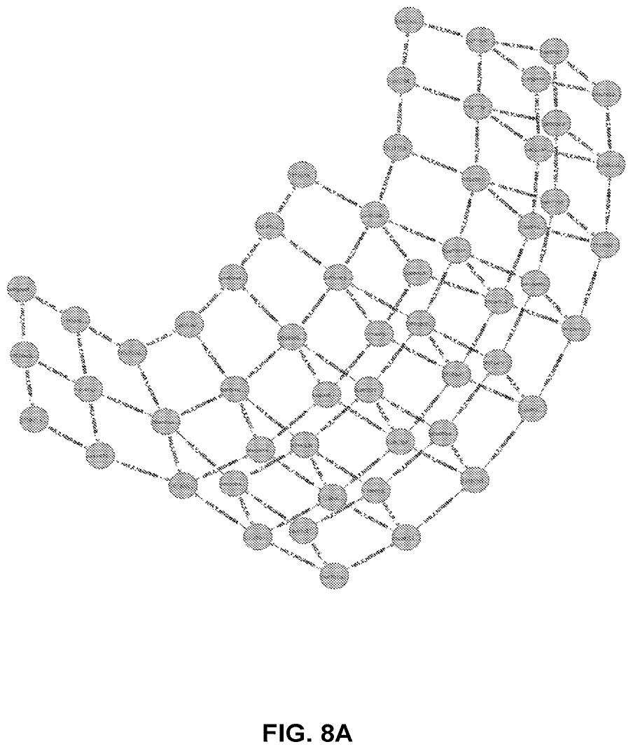

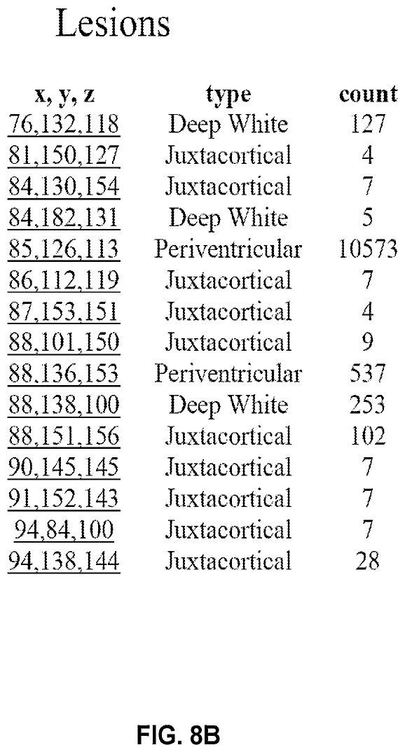

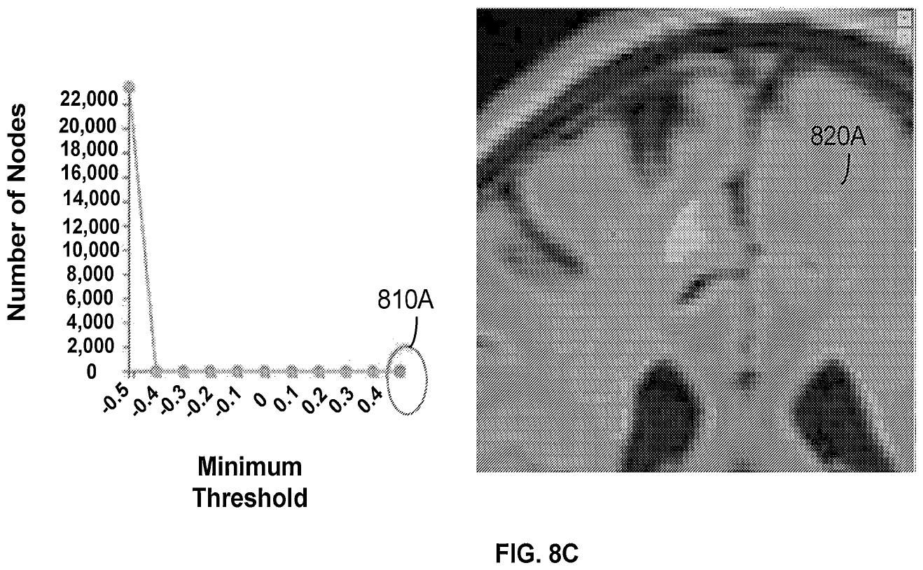

In such embodiments, based on a set minimum threshold, adjacent nodes are iteratively interrogated, thereby generating a node neighborhood. In various embodiments, the set minimum threshold can be altered to generate additional node neighborhoods. For example, the set minimum threshold can be incremented or decremented by a fixed value, and adjacent nodes can be iteratively interrogated to generate an additional node neighborhood. For example, a set minimum threshold can be incremented or decremented by fixed values of any one of 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1.0. Thus, different node neighborhoods can be generated based on each minimum threshold. By generating different node neighborhoods based on different minimum thresholds, this enables subsequent display, visualization, and transitioning between the different node neighborhoods according to the minimum thresholds, as is described further below in FIGS. 8C and 8D.

In various embodiments, the interrogation of each node can be conducted on a per-anatomical location basis using anatomical information encoded in the node. For example, the embodiments described regarding the interrogation of each node can be conducted within individual anatomical locations. For example, a seed node is identified within an anatomical location and therefore, a minimum threshold is established for the anatomical location. Therefore, interrogation of a node within an anatomical location can be conducted by comparing signal intensity information of the node to a minimum threshold value of the specific anatomical location. Additionally, the interrogation of a node within a different anatomical location can be conducted by comparing signal intensity information of the node to a minimum threshold value of the different anatomical location.

In various embodiments, when interrogating a particular node for inclusion or exclusion from a node neighborhood, the particular node's anatomical location is compared to the anatomical locations of nodes in the node neighborhood. For example, in response to determining that the particular node's anatomical location does not differ from the anatomical location of a node in the node neighborhood, the particular node is included in the node neighborhood. As another example, in response to determining that the particular node's anatomical location differs from the anatomical location of a node in the node neighborhood, then the particular node is excluded from the node neighborhood.

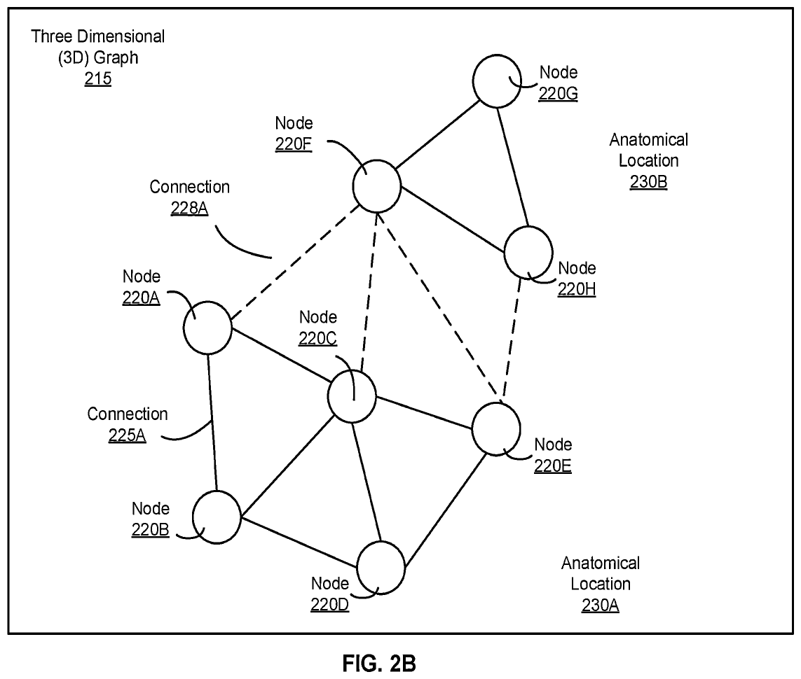

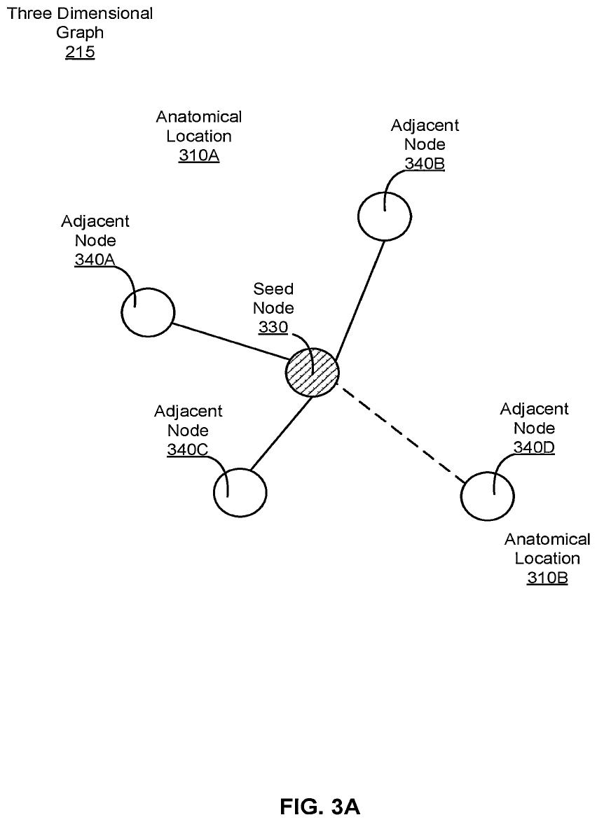

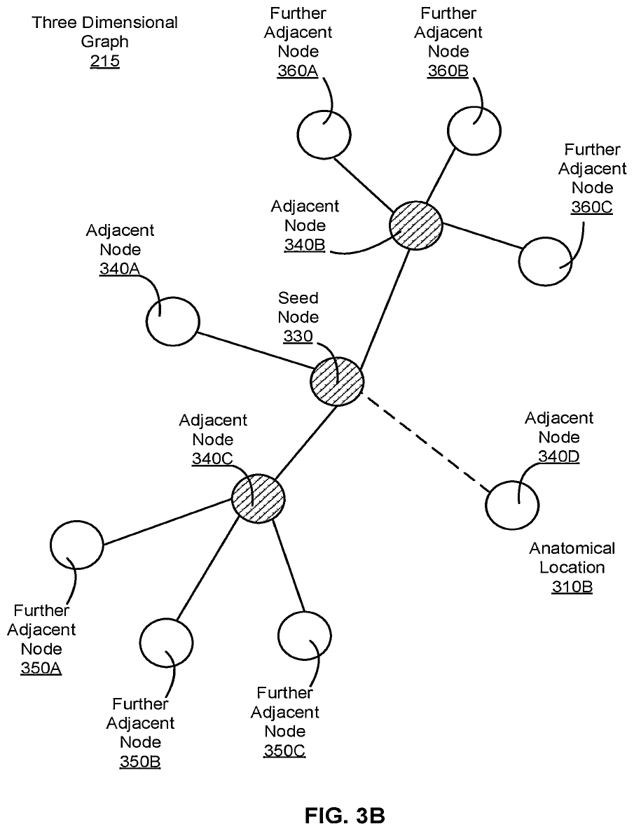

Reference is now made to FIGS. 3A-3C which together depict the identification of a node neighborhood indicative of an anatomical abnormality. Specifically, FIG. 3A depicts a first step of determining a node neighborhood involving the identification of a seed node, in accordance with an embodiment. In particular, FIG. 3A includes a seed node 330, three adjacent nodes 340A, 340B, and 340C that are in the same anatomical location 310A as seed node 330, as well as one adjacent node 340D that is in a different anatomical location 310B. Although not shown in FIG. 3A, there may be additional nodes (e.g., additional adjacent nodes to the seed node 330 as well as additional nodes adjacent to any of the adjacent nodes 340A, 340B, 340C, and 340D). At this stage, a seed node 330 has been identified and included in a node neighborhood. Next, the adjacent nodes 340A, 340B, 340C, and 340D are individually interrogated for inclusion or exclusion in the node neighborhood based on signal intensity information and/or anatomical information encoded in each node. For example, adjacent nodes 340A, 340B, and 340C can be interrogated based on a first minimum threshold value for the first anatomical location 310A and adjacent node 340D can be interrogated based on a second minimum threshold value for the second anatomical location 310B.

Reference is now made to FIG. 3B, which depicts a second step of determining a node neighborhood involving the interrogation of adjacent nodes, in accordance with an embodiment. Here, adjacent node 340A and adjacent node 340D are excluded from the node neighborhood. Therefore further adjacent nodes that may be connected to adjacent node 340A and adjacent node 340D (not shown in FIG. 3B) are not further interrogated. Additionally, the interrogation of adjacent node 340B and adjacent node 340C resulted in their inclusion in the node neighborhood (as indicated by their dashed fill in FIG. 3B). neighborhood. Thus, further adjacent nodes that are connected to adjacent node 340C and adjacent node 340B are further individually interrogated according to the methods described herein. These include further adjacent nodes 350A, 350B, 350C, 360A, 360B, and 360C.

Here, assume that interrogation of each of the further adjacent nodes 350A, 350B, 350C, 360A, 360B, and 360C resulted in exclusion of all the further adjacent nodes. Thus, given that the iterative interrogation of nodes has concluded, the node neighborhood is generated. Reference is now made to FIG. 3C, which depicts an example node neighborhood indicative of an anatomical abnormality, in accordance with the embodiments shown in FIGS. 3A and 3B. Here, the node neighborhood 370 includes the seed node 330, adjacent node 340B, and adjacent node 340C. Additionally, FIG. 3C is one example of a representation of the node neighborhood 370. In various embodiments, the node neighborhood 370 can be projected into the 3D graph for display and/or visualization purposes. In various embodiments, the node neighborhood 370 can be overlaid and displayed on top of MRI brain scan images, thereby enabling visualization of the node neighborhood 370 in relation to the MRI brain scan images.

In various embodiments, the representation of the node neighborhood 370 is stored (e.g., stored in the graph store 170 shown in FIG. 1B). In one embodiment, the node neighborhood 370 is stored by encoding in the nodes of the node neighborhood information that indicates their inclusion in a node neighborhood. For example, referring again to node neighborhood 370 in FIG. 3C, each of seed node 330, adjacent node 340B, and adjacent node 340C can be encoded with information identifying their inclusion in node neighborhood 370. Storing the node neighborhood 370 enables the subsequent retrieval of the node neighborhood 370 for analysis of temporal changes of the anatomical abnormality and/or visualization of the changes of the anatomical abnormality, as is described in further detail below.

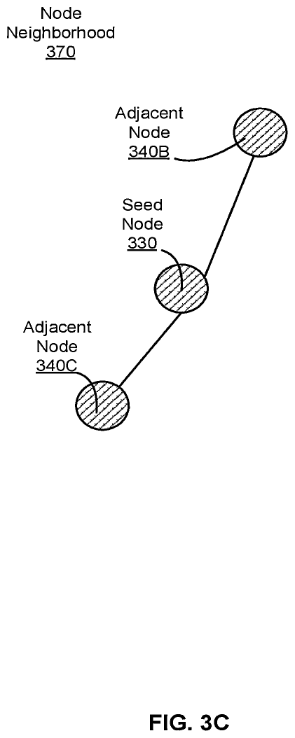

FIG. 4 is a flow process 405 for generating a representation of an anatomical abnormality in a 3D graph, in accordance with an embodiment. Step 410 involves obtaining a set of images comprising an anatomical abnormality. Step 420 involves generating a 3D graph using at least the set of images, the 3D graph comprising a plurality of nodes. Generally, each node in the 3D graph is encoded with information such as any of signal intensity information, spatial information, neighbor node information, temporal information, and anatomical information.

Step 430 involves defining a node neighborhood indicative of the anatomical abnormality within the 3D graph. Here, defining the node neighborhood involves an iterative process involving at least steps 440A and 440B. Specifically, step 440A involves interrogating adjacent nodes of the seed node for inclusion in the node neighborhood. Step 440B involves interrogating further adjacent nodes for inclusion in the node neighborhood. Here, the further adjacent nodes are connected to adjacent nodes that have been included in the node neighborhood. Although not shown, if further adjacent nodes are included in the node neighborhood, the iterative interrogation process can continue for further nodes that are connected to any node that has been included in the node neighborhood.

Step 450 involves generating a representation of the anatomical abnormality within the 3D graph.

### V. Example Process for Updating an Anatomical Abnormality Within a 3D Graph

Embodiments disclosed herein involve the generation of a 3D graph and identifying an anatomical abnormality within the 3D graph. Additionally, the anatomical abnormality can be further updated within the 3D graph. For example, a first set of images can be captured and used to identify an anatomical abnormality within the 3D graph, as described above. Here, the set of images may be captured from a subject at a first timepoint. Thus, the identified anatomical abnormality corresponds to the first timepoint. Next, a second set of images can be captured from the subject at a second timepoint. Thus, the second set of images can be used to identify the same anatomical abnormality within the 3D graph, thereby providing a representation of the anatomical abnormality at the second timepoint. Thus, the representations of the anatomical abnormality at the first timepoint and the second timepoint enables a temporal understanding of the anatomical abnormality (e.g., how the anatomical abnormality is changing across the timepoints). In various embodiments, further representations of the anatomical abnormality can be generated at subsequent timepoints (e.g., third timepoint, fourth timepoint, etc.) according to the methods described herein.

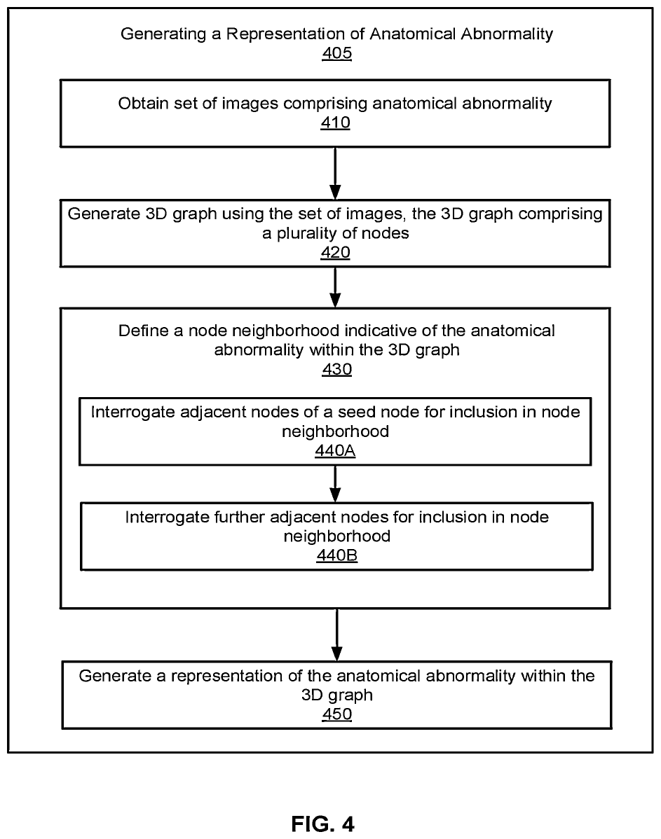

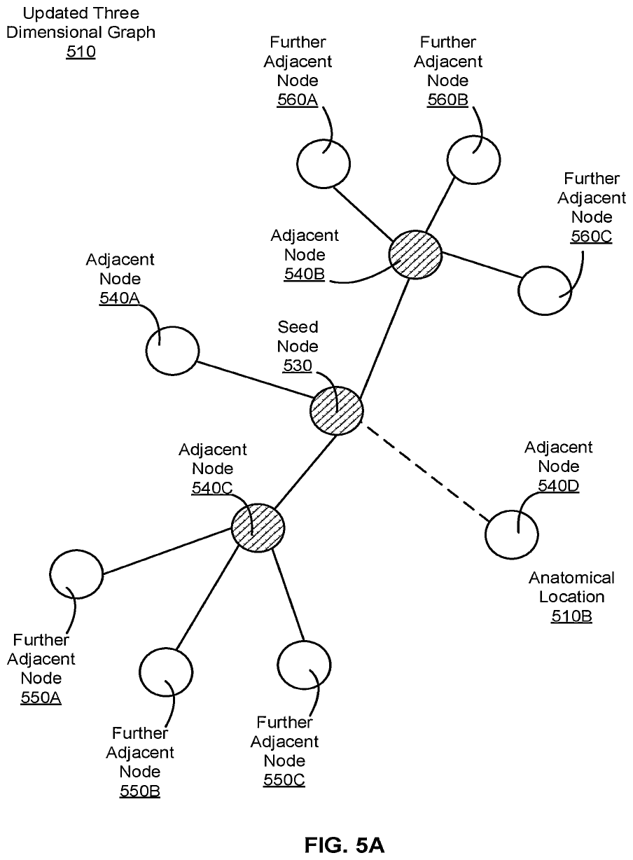

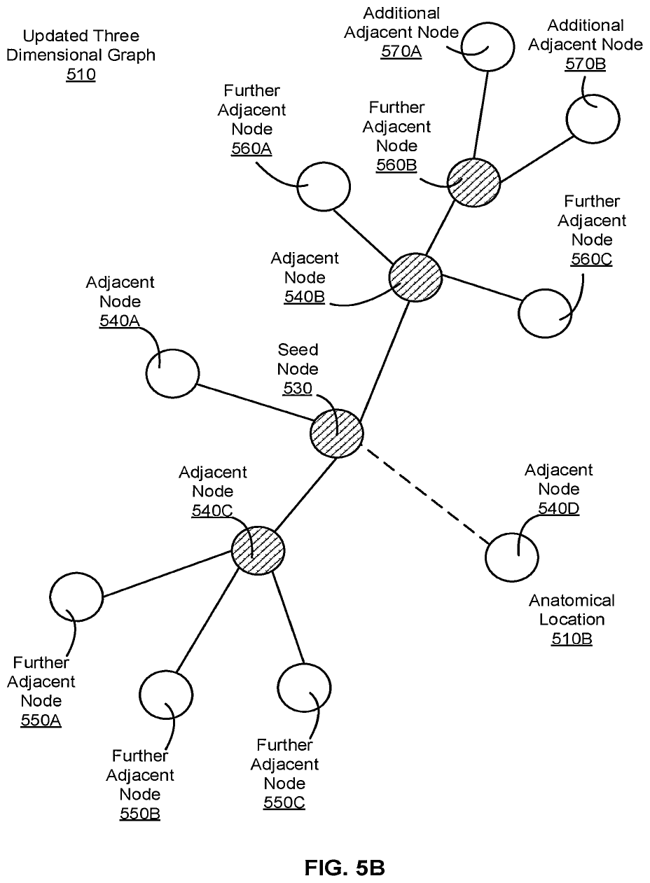

Reference is now made to FIG. 5A, which depicts the implementation of an updated three dimensional graph for determining a temporal change of the anatomical abnormality, in accordance with an embodiment. Here, the updated three dimensional graph 510 may be generated from a second set of images captured from the subject at a second timepoint and therefore, the updated three dimensional graph 510 is a representation corresponding to the second timepoint. Notably, FIG. 5A depicts a region in the updated 3D graph 510 that corresponds to the region of the 3D graph 215 shown in FIGS. 3A-3B.

To arrive at the updated three dimensional graph 510 in FIG. 5A, the seed node 530 is first identified and included in the node neighborhood. Next, adjacent nodes 540A, 540B, 540C, and 540D are individually interrogated for inclusion or exclusion from the node neighborhood. Here, adjacent node 540B and adjacent node 540C are included in the node neighborhood whereas adjacent node 540A and adjacent node 540D are excluded. Next, each of the further adjacent nodes (e.g., further adjacent nodes 550A, 550B, 550C, 560A, 560B, and 560C) are analyzed.

Reference is now made to FIG. 5B, which depicts the interrogation of additional nodes in the updated three dimensional graph for determining a temporal change of the anatomical abnormality, in accordance with an embodiment. Here, the interrogation of the further adjacent nodes 550A, 550B, 550C, 560A, and 560C resulted in exclusion of those further adjacent nodes. Additionally, interrogation of the further adjacent node 560B resulted in its inclusion in the node neighborhood. Thus, additional adjacent nodes 570A and 570B are further interrogated given that they are connected to further adjacent node 560B. In this example, additional adjacent node 570A and additional adjacent node 570B are excluded from the node neighborhood.

Reference is now made to FIG. 5C, which depicts an example updated node neighborhood indicative of an anatomical abnormality, in accordance with the embodiments shown in FIGS. 5A and 5B. Here, FIG. 5C shows a representation of the anatomical abnormality corresponding to the second set of images captured from the subject at the second timepoint. Specifically, the updated node neighborhood 580 includes each of the seed node 530, adjacent node 540B, adjacent node 540C, and further adjacent node 560B.

In various embodiments, the representation of the updated node neighborhood 580 is stored (e.g., stored in the graph store 170 shown in FIG. 1B). In one embodiment, the updated node neighborhood 580 is stored by encoding in the nodes of the node neighborhood information that indicates their inclusion in a node neighborhood. For example, referring again to updated node neighborhood 580 in FIG. 5C, each of seed node 530, adjacent node 540B, adjacent node 540C, and further adjacent node 560B can be encoded with information identifying their inclusion in updated node neighborhood 580. Storing the updated node neighborhood 580 enables the subsequent retrieval for analysis of temporal changes of the anatomical abnormality and/or visualization of the changes of the anatomical abnormality.

### V. Characterizing an Anatomical Abnormality Using the 3D Graph

Embodiments disclosed herein involving identifying anatomical abnormalities within 3D graphs at one or more multiple timepoints. Using the anatomical abnormalities within the 3D graphs, the anatomical abnormalities can be characterized using the 3D graph. Generally, the steps described here for characterizing an anatomical abnormality (e.g., a lesion) can be performed by the disease characterization module 150 described above in reference to FIG. 1B.

In various embodiments, the disease characterization module 150 obtains the one or more representations of the anatomical abnormalities across the one or more timepoints (e.g., retrieves from graph store 170 shown in FIG. 1B) and analyzes the one or more representations of the anatomical abnormalities. For example, a representation of the anatomical abnormality may be a node neighborhood comprising a plurality of nodes. This analysis reveals topological features and/or temporal changes of the disease.

In some embodiments, the disease characterization module 150 obtains one representation of the anatomical abnormality and characterizes features of the disease based on the one representation of the anatomical abnormality. For example, the disease characterization module 160 may determine a disease characterization based on single timepoint characteristics of the anatomical abnormality, including inter or intra-abnormality relationships, abnormality adjacency to anatomical landmarks, intra-abnormality voids (e.g., as a measure of tissue damage within an abnormality), separated abnormality surfaces from internal components, abnormality characteristics (e.g., surface, texture, shape, topology, density, homogeneity), abnormality volumetrics (e.g., total abnormality load).

In some embodiments, the disease characterization module 150 obtains two or more representations of the anatomical abnormality and characterizes features of the disease based on the two or more representations of the anatomical abnormality. Here, the disease characterization module 160 may determine the change in the anatomical abnormality across two or more timepoints. The disease characterization module 150 may compare information encoded in the nodes of the first representation of the anatomical abnormality to information encoded in the nodes of the second representation of the anatomical abnormality to validate that both representations correspond to the same anatomical abnormality. For example, the disease characterization module 150 can compare the spatial information (e.g., x, y, and z coordinates) of nodes in the representations to validate that both representations correspond to the same anatomical abnormality.

To determine the change in the anatomical abnormality across two or more timepoints, the disease characterization module 150 can compare the node neighborhood of the anatomical abnormality for the first timepoint to the node neighborhood of the anatomical abnormality for the second timepoint. This comparison reveals the change of the anatomical abnormality across the first and second timepoints. The changes in the anatomical abnormality can include a change in inter or intra-abnormality relationships, change in abnormality adjacency to anatomical landmarks, change in intra-abnormality voids (e.g., as a measure of tissue damage within an abnormality), change in separated abnormality surfaces from internal components, change in abnormality characteristics (e.g., change in any of surface, texture, shape, topology, density, homogeneity), change in abnormality volumetrics (e.g., change in total abnormality load, merging or splitting abnormalities).

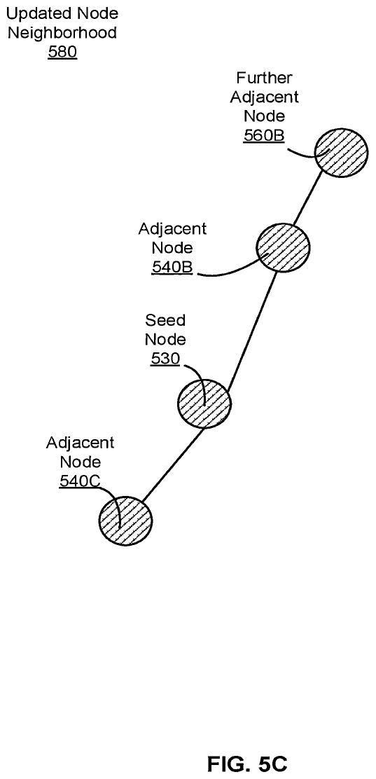

Reference is now made to FIG. 6, which depicts an example transition between the node neighborhood and updated node neighborhood, in accordance with an embodiment. The node neighborhood 370 is described above in reference to FIG. 3C and the updated node neighborhood 580 is described above in reference to FIG. 5C.

In various embodiments, the disease characterization module 150 can analyze each of the node neighborhood 370 and the updated node neighborhood 580 separately and characterizes the disease at each timepoint based on single timepoint characteristics of the anatomical abnormality. In various embodiments, the disease characterization module 150 analyzes the node neighborhood 370 and the updated node neighborhood 580 together to determine changes of the anatomical abnormality over the timepoints. In this particular example, the disease characterization module 150 can determine that the updated node neighborhood 580 additionally includes further adjacent node 560B whereas that node is missing in the node neighborhood 370. The disease characterization module 150 can further quantify the number of nodes in each node neighborhood (e.g., 3 nodes in the node neighborhood 370 and 4 nodes in the updated node neighborhood 580). Here, the disease characterization module 150 can determine that the anatomical abnormality is increasing in size (e.g., due to increasing number of nodes in the node neighborhood). Thus, the disease characterization module 150 may determine that the disease is progressing in the subject.

In various embodiments, the disease characterization module 150 may display one or more representations of anatomical abnormalities. This enables visualization of the anatomical abnormality and/or visualization of the temporal changes to the anatomical abnormality. For example, returning again to FIG. 6, the disease characterization module 150 may display node neighborhood 370 and updated node neighborhood 580 and furthermore, may display a transition from the display node neighborhood 370 to the updated node neighborhood 580. This enables visualization of the changing anatomical abnormality across the different timepoints. For example, the node neighborhood 370 and updated node neighborhood 580 can be displayed to a user, such that the user can visually interpret the change to the anatomical abnormality across the two timepoints.

### VI. Example Diseases and Anatomical Abnormalities

Methods described herein involve generating and implementing 3D graph models for subjects that are useful for characterizing diseases. Example diseases can include, but are not limited to, any of neurodegenerative diseases, neurological diseases, oncologies (e.g., cancers), cardiovascular diseases, or pulmonary diseases.

In various embodiments, the disease is a neurodegenerative disease. In such embodiments, a neurodegenerative disease can be characterized by anatomical abnormalities, such as one or more lesions or atrophy.

In various embodiments, the neurodegenerative disease or neurological disease is any one of Multiple Sclerosis (MS), Alzheimer's disease, Parkinson's disease, traumatic CNS injury, Down Syndrome (DS), glaucoma, amyotrophic lateral sclerosis (ALS), frontotemporal dementia (FTD), and Huntington's disease. In various embodiments, the neurodegenerative or neurological disease is any one of Absence of the Septum Pellucidum, Acid Lipase Disease, Acid Maltase Deficiency, Acquired Epileptiform Aphasia, Acute Disseminated Encephalomyelitis, ADHD, Adie's Pupil, Adie's Syndrome, Adrenoleukodystrophy, Agenesis of the Corpus Callosum, Agnosia, Aicardi Syndrome, AIDS, Alexander Disease, Alper's Disease, Alternating Hemiplegia, Anencephaly, Aneurysm, Angelman Syndrome, Angiomatosis, Anoxia, Antiphosphipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Autism, Autonomic Dysfunction, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Injuries, Bradbury-Eggleston Syndrome, Brain or Spinal Tumors, Brain Aneurysm, Brain injury, Brown-Sequard Syndrome, Bulbospinal Muscular Atrophy, Cadasil, Canavan Disease, Causalgia, Cavernomas, Cavernous Angioma, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysm, Cerebral Arteriosclerosis, Cerebral Atrophy, Cerebral Beriberi, Cerebral Gigantism, Cerebral Hypoxia, Cerebral Palsy, Cerebro-Oculo-Facio-Skeletal Syndrome, Charcot-Marie-Tooth Disease, Chiari Malformation, Chorea, Chronic Inflammatory Demyelinating Polyneuropathy (CIDP), Coffin Lowry Syndrome, Colpocephaly, Congenital Facial Diplegia, Congenital Myasthenia, Congenital Myopathy, Corticobasal Degeneration, Cranial Arteritis, Craniosynostosis, Creutzfeldt-Jakob Disease, Cumulative Trauma Disorders, Cushing's Syndrome, Cytomegalic Inclusion Body Disease, Dancing Eyes-Dancing Feet Syndrome, Dandy-Walker Syndrome, Dawson Disease, Dementia, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyssynergia Cerebellaris Myoclonica, Dystonias, Early Infantile Epileptic Encephalopathy, Empty Sella Syndrome, Encephalitis, Encephalitis Lethargica, Encephaloceles, Encephalopathy, Encephalotrigeminal Angiomatosis, Epilepsy, Erb-Duchenne and Dejerine-Klumpke Palsies, Erb's Palsy, Essential Tremor, Extrapontine Myelinolysis, Fabry Disease, Fahr's Syndrome, Fainting, Familial Dysautonomia, Familial Hemangioma, Familial Periodic Paralyzes, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Hemicrania Continua, Hemifacial Spasm, Hemiplegia Alterans, Hereditary Neuropathy, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydromyelia, Hypernychthemeral Syndrome, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaac's Syndrome, Joubert syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus, Lyme Disease, Machado-Joseph Disease, Macrencephaly, Melkersson-Rosenthal Syndrome, Meningitis, Menkes Disease, Meralgia Paresthetica, Metachromatic Leukodystrophy, Microcephaly, Migraine, Miller Fisher Syndrome, Mini-Strokes, Mitochondrial Myopathies, Motor Neuron Diseases, Moyamoya Disease, Mucolipidoses, Mucopolysaccharidoses, Multiple sclerosis (MS), Multiple System Atrophy, Muscular Dystrophy, Myasthenia Gravis, Myoclonus, Myopathy, Myotonia, Narcolepsy, Neuroacanthocytosis, Neurodegeneration with Brain Iron Accumulation, Neurofibromatosis, Neuroleptic Malignant Syndrome, Neurosarcoidosis, Neurotoxicity, Nevus Cavernosus, Niemann-Pick Disease, Non 24 Sleep Wake Disorder, Normal Pressure Hydrocephalus, Occipital Neuralgia, Occult Spinal Dysraphism Sequence, Ohtahara Syndrome, Olivopontocerebellar Atrophy, Opsoclonus Myoclonus, Orthostatic Hypotension, O'Sullivan-McLeod Syndrome, Overuse Syndrome, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Perineural Cysts, Periodic Paralyzes, Peripheral Neuropathy, Periventricular Leukomalacia, Pervasive Developmental Disorders, Pinched Nerve, Piriformis Syndrome, Plexopathy, Polymyositis, Pompe Disease, Porencephaly, Postherpetic Neuralgia, Postinfectious Encephalomyelitis, Post-Polio Syndrome, Postural Hypotension, Postural Orthostatic Tachyardia Syndrome (POTS), Primary Lateral Sclerosis, Prion Diseases, Progressive Multifocal Leukoencephalopathy, Progressive Sclerosing Poliodystrophy, Progressive Supranuclear Palsy, Prosopagnosia, Pseudotumor Cerebri, Ramsay Hunt Syndrome I, Ramsay Hunt Syndrome II, Rasmussen's Encephalitis, Reflex Sympathetic Dystrophy Syndrome, Refsum Disease, Refsum Disease, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Saint Vitus Dance, Sandhoff Disease, Schizencephaly, Septo-Optic Dysplasia, Shingles, Shy-Drager Syndrome, Sjogren's Syndrome, Sleep Apnea, Sleeping Sickness, Sotos Syndrome, Spasticity, Spinal Cord Infarction, Spinal Cord Injury, Spinal Cord Tumors, Spinocerebellar Atrophy, Spinocerebellar Degeneration, Stiff-Person Syndrome, Striatonigral Degeneration, Stroke, Sturge-Weber Syndrome, SUNCT Headache, Syncope, Syphilitic Spinal Sclerosis, Syringomyelia, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tinnitus, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis including Temporal Arteritis, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson's Disease, Wolman's Disease, X-Linked Spinal and Bulbar Muscular Atrophy, and Zellweger Syndrome.

In various embodiments, the disease is a cancer. In such embodiments, a cancer can be characterized by anatomical abnormalities, such as one or more tumor masses.

In various embodiments, the cancer can include one or more of: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, stomach cancer, thyroid cancer, head and neck carcinoma, large bowel cancer, hematopoietic cancer, testicular cancer, colon and/or rectal cancer, uterine cancer, or prostatic cancer. In some embodiments, the cancer in the subject can be a metastatic cancer, including any one of bladder cancer, breast cancer, colon cancer, kidney cancer, lung cancer, melanoma, ovarian cancer, pancreatic cancer, prostatic cancer, rectal cancer, stomach cancer, thyroid cancer, or uterine cancer.

### VII. Guided Decision Making Using the 3D Graph

Embodiments described herein involve determining a disease characterization for a subject by using a 3D graph, the disease characterization indicating topological features and/or temporal changes of the disease. In various embodiments, the disease characterization is useful for performing a differential diagnosis of the disease. For example, in a scenario where the subject has not yet been diagnosed with the disease, the disease characterization can reveal the presence of one or more anatomical abnormalities that are indicative of the presence of disease. Thus, the disease characterization can be used to diagnose the subject with the disease.

In various embodiments, the disease characterization is useful for determining an efficacy of a therapy previously administered to the individual. For example, the subject may already be administered a therapy. Thus, the disease characterization can reveal whether the therapy is effective in treating the disease (e.g., reversing the disease or eliminating the disease) based on the topological features or temporal changes of one or more anatomical abnormalities that are indicative of the disease.

In various embodiments, the disease characterization is useful for selecting a therapy (e.g., a candidate therapy) for the individual. For example, the disease characterization may reveal that the disease has progressed or is continuing to progress as evidenced by the topological features or temporal changes of one or more anatomical abnormalities. Thus, a therapy that is approved to treat the disease in the progressed state can be selected. In various embodiments, a selected therapy can include one or more of a biologic, e.g. a cytokine, antibody, soluble cytokine receptor, anti-sense oligonucleotide, siRNA, etc. Such biologic agents encompass muteins and derivatives of the biological agent, which derivatives can include, for example, fusion proteins, PEGylated derivatives, cholesterol conjugated derivatives, and the like as known in the art. Also included are antagonists of cytokines and cytokine receptors, e.g. traps and monoclonal antagonists, e.g. IL-1Ra, IL-1 Trap, sIL-4Ra, etc. Also included are biosimilar or bioequivalent drugs to the active agents set forth herein.

Example therapies for multiple sclerosis include corticosteroids, plasma exchange, ocrelizumab (Ocrevus®), IFN-β (Avonex®, Betaseron®, Rebif®, Extavia®, Plegridy®), Glatiramer acetate (Copaxone®, Glatopa®), anti-VLA4 (Tysabri, natalizumab), dimethyl fumarate (Tecfidera®, Vumerity®), teriflunomide (Aubagio®), monomethyl fumarate (Bafiertam™), ozanimod (Zeposia®), siponimod (Mayzent®), fingolimod (Gilenya®), anti-CD52 antibody (e.g., alemtuzumab (Lemtrada®), mitoxantrone (Novantrone®), methotrexate, cladribine (Mavenclad®, simvastatin, and cyclophosphamide. In addition or alternative to therapeutic agents, other therapies for multiple sclerosis include lifestyle changes such as physical therapy or a change in diet. The method also provides for combination therapies of one or more therapeutic agents and/or additional treatments, where the combination can provide for additive or synergistic benefits.

In various embodiments, a pharmaceutical composition can be selected and/or administered to the subject based on the disease characterization, the selected therapeutic agent likely to exhibit efficacy against the disease. A pharmaceutical composition administered to an individual includes an active agent such as the therapeutic agent described above. The active ingredient is present in a therapeutically effective amount, i.e., an amount sufficient when administered to treat a disease or medical condition mediated thereby. The compositions can also include various other agents to enhance delivery and efficacy, e.g. to enhance delivery and stability of the active ingredients. Thus, for example, the compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents. The composition can also include any of a variety of stabilizing agents, such as an antioxidant.

The pharmaceutical compositions or therapeutic agents described herein can be administered in a variety of different ways. Examples include administering a composition containing a pharmaceutically acceptable carrier via oral, intranasal, intramodular, intralesional, rectal, topical, intraperitoneal, intravenous, intramuscular, subcutaneous, subdermal, transdermal, intrathecal, endobronchial, transthoracic, or intracranial method.

### VIII. Computer Implementation

The methods of the invention, including the methods of generating and implementing a 3D graph, are, in some embodiments, performed on one or more computers.

For example, the building and deployment of a 3D graph can be implemented in hardware or software, or a combination of both. In one embodiment of the invention, a machine-readable storage medium is provided, the medium comprising a data storage material encoded with machine readable data which, when using a machine programmed with instructions for using said data, is capable of building and implementing a 3D graph and/or displaying any of the datasets or results described herein. The invention can be implemented in computer programs executing on programmable computers, comprising a processor, a data storage system (including volatile and non-volatile memory and/or storage elements), a graphics adapter, a pointing device, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g. word processing text file, database format, etc.

In some embodiments, the methods of the invention, including the methods for generating or implementing a 3D graph, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 7 illustrates an example computer for implementing the entities shown in FIGS. 1A and 1B. The computer 700 includes at least one processor 702 coupled to a chipset 704. The chipset 704 includes a memory controller hub 720 and an input/output (I/O) controller hub 722. A memory 706 and a graphics adapter 712 are coupled to the memory controller hub 720, and a display 718 is coupled to the graphics adapter 712. A storage device 708, an input device 714, and network adapter 716 are coupled to the I/O controller hub 722. Other embodiments of the computer 700 have different architectures.

The storage device 708 is a non-transitory computer-readable storage medium such as a hard drive, compact disk read-only memory (CD-ROM), DVD, or a solid-state memory device. The memory 706 holds instructions and data used by the processor 702. The input interface 714 is a touch-screen interface, a mouse, track ball, or other type of pointing device, a keyboard, or some combination thereof, and is used to input data into the computer 700. In some embodiments, the computer 700 may be configured to receive input (e.g., commands) from the input interface 714 via gestures from the user. The network adapter 716 couples the computer 700 to one or more computer networks.

The graphics adapter 712 displays images and other information on the display 718. In various embodiments, the display 718 is configured such that the user may input user selections on the display 718 to, for example, generate a 3D graph including one or more anatomical abnormalities. In one embodiment, the display 718 may include a touch interface. In various embodiments, the display 718 can show representations (e.g., node neighborhoods) of one or more anatomical abnormalities. In various embodiments, the display 718 can show representations of one or more anatomical abnormalities overlaid on images, such as MRI images, thereby enabling the visualization of the anatomical abnormalities on the images. In various embodiments, the display 718 can show transitions between representations (e.g., node neighborhoods) of anatomical abnormalities across multiple timepoints, thereby enabling visualization of the temporal changes of the anatomical abnormalities.

The computer 700 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 708, loaded into the memory 706, and executed by the processor 702.

The types of computers 700 used by the entities of FIG. 1A or 1B can vary depending upon the embodiment and the processing power required by the entity. For example, the graph system 130 can run in a single computer 700 or multiple computers 700 communicating with each other through a network such as in a server farm. The computers 700 can lack some of the components described above, such as graphics adapters 712, and displays 718.

### IX. Systems

Further disclosed herein are systems for implementing 3D graphs. In various embodiments, such a system can include at least the graph system 130 described above in FIG. 1A. In various embodiments, the graph system 130 is embodied as a computer system, such as a computer system with example computer 700 described in FIG. 7.

In various embodiments, the system includes an imaging device, such as an imaging generation system 120 described above in FIG. 1A. In various embodiments, the system includes both the graph system 130 (e.g., a computer system) and an imaging generation system 120. In such embodiments, the graph system 130 can be communicatively coupled with the image generation system 120 to receive images captured from a subject. Thus, the graph system 130 builds and implements, in silico, 3D graphs for revealing topology and temporal nature of diseases.

### Additional Embodiments

Embodiments disclosed herein describe the generation and implementation of a 3D graph developed from images captured from patients with a disease. Such a 3D graph is useful for analyzing diseases in patients (e.g., disease risk or disease progression). As one example, images captured from patients can be brain images and as such, the 3D graph is useful for analyzing disease risk and/or disease progression of neurodegenerative diseases (e.g., multiple sclerosis (MS), amyotrophic lateral sclerosis (ALS), or chronic inflammatory demyelinating polyneuropathy (CIDP)). As another example, images captured from patients can be images of other organs (e.g., thorax, lung, abdomen, colon, cervix, pancreas, kidney, liver) and therefore, 3D graphs generated from these images are useful for analyzing disease risk and/or disease progression of non-neurodegenerative diseases (e.g., oncologies, cardiovascular diseases, pulmonary diseases, etc.) that involve the particular organ that has been imaged. Example images captured from patients with a disease include magnetic resonance images (MRI), computed tomography (CT) images, positron emission tomography (PET) images, and X-ray radiography.

In particular embodiments, a 3D graph is generated from brain MRI images captured from patients (e.g., multiple sclerosis (MS) patients). Novel visualization of neuroimaging data can lead to clinical insights and ultimately new imaging analysis capabilities. Graph models of magnetic resonance imaging (MRI) data can reveal the topology and temporal nature of multiple sclerosis disease progression, by exposing novel structural features of the brain through representation of data as interactive 3D projections. Existing standards and evolving approaches to neuroimaging can benefit from an integration of graph analytics and visualization.

In one aspect, the disclosure provides a method comprising obtaining a first set of brain images and a second set of brain images each comprising a lesion, the first and second sets of brain images captured from a MS patient at a first timepoint and second timepoint, respectively; for each of the first set of brain images and second set of brain images, generating a 3D image by: extracting a lesion community of nodes using at least spatial characteristics of individual voxels, the lesion community comprising nodes corresponding to the lesion; generating a 3D graph of the lesion by connecting the lesion community of nodes of the 3D image derived from the first set of brain images to the lesion community of nodes of the 3D image derived from the second set of brain images.

In various embodiments, the method further comprises assessing a change or non-change of MS disease activity in the MS patient using the 3D graph. In various embodiments, the MS disease activity is any one of: inter or intralesion relationships, lesion adjacency to neuroanatomy, intralesion voids (e.g., as a measure of permanent tissue damage), separated lesion surfaces from internal components, lesion characteristics (e.g., lesion surface, texture, shape, topology, density, homogeneity), temporal changes to lesions (e.g., new lesion, enlarging lesion, or shrinking lesion), and lesion volumetrics (e.g., total lesion load, merging, or splitting lesions).

In various embodiments, the method further comprises: based on the assessment of the change or non-change of MS disease activity, performing one or more of: performing a differential diagnosis of the patient's MS; selecting a candidate therapy for the patient; and determining an efficacy of a therapy previously administered to the patient. In various embodiments, the first set of brain images and second set of brain images are MRI images. In various embodiments, extracting a lesion community of nodes using at least spatial characteristics of individual voxels further comprises: performing a thresholding to identify candidate nodes to be included in the lesion community, the candidate nodes satisfying a specified threshold condition.

In another aspect, the disclosure provides a non-transitory computer-readable storage medium storing computer program instructions that when executed by a computer processor, cause the computer processor to perform any combination of the method steps mentioned above.

In another aspect, the disclosure provides a system that includes a storage memory and a processor communicatively coupled to the storage memory. The storage memory is configured to store image data, such as brain MRI images obtained from patients. The processor is configured to perform any combination of the method steps mentioned above. In some embodiments, the processor can be further configured to assess a change or non-change of MS disease activity in the MS patient using the 3D graph, as discussed above. In some embodiments, based on the assessment of the change or non-change of MS disease activity, the processor can be further configured to perform the steps of any one or more of: performing a differential diagnosis of the patient's MS; selecting a candidate therapy for the patient; and determining an efficacy of a therapy previously administered to the patient.

Additionally disclosed herein is a method comprising: obtaining a first set of brain images and a second set of brain images each comprising a lesion, the first and second sets of brain images captured from a multiple sclerosis (MS) patient at a first timepoint and second timepoint, respectively; for each of the first set of brain images and second set of brain images, generating a multi-dimensional image, optionally a three dimensional (3D) image by: extracting a lesion community of nodes using at least spatial characteristics of individual voxels, the lesion community comprising nodes corresponding to the lesion; generating a multi-dimensional graph of the lesion by connecting the lesion community of nodes of the multi-dimensional image derived from the first set of brain images to the lesion community of nodes of the multi-dimensional image derived from the second set of brain images. In various embodiments, methods disclosed herein further comprise assessing a change or non-change of MS disease activity in the MS patient using the multi-dimensional graph. In various embodiments, the MS disease activity is any one of: inter or intralesion relationships, lesion adjacency to neuroanatomy, intralesion voids (e.g., as a measure of permanent tissue damage), separated lesion surfaces from internal components, lesion characteristics (e.g., lesion surface, texture, shape, topology, density, homogeneity), temporal changes to lesions (e.g., new lesion, enlarging lesion, or shrinking lesion), and lesion volumetrics (e.g., total lesion load, merging, or splitting lesions).

In various embodiments, methods disclosed herein further comprise: based on the assessment of the change or non-change of MS disease activity, performing one or more of: performing a differential diagnosis of the patient's MS; selecting a candidate therapy for the patient; and determining an efficacy of a therapy previously administered to the patient. In various embodiments, the first set of brain images and second set of brain images are MRI images. In various embodiments, extracting a lesion community of nodes using at least spatial characteristics of individual voxels further comprises: performing a thresholding to identify candidate nodes to be included in the lesion community, the candidate nodes satisfying a specified threshold condition.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used, but some experimental error and deviation should be allowed for.

### Example 1: Developing Interactive 3D Graph Representation of MRI Data from MS Patients

In this example, the goal was to develop a cloud-based workflow to translate Digital Imaging and Communications in Medicine (DICOM) imaging data files into a visual, interactive graph schema. The resulting application enhances and supports the current evaluation of disease features on conventional MRI and reveals the temporal features of lesion and disease progression in patients with multiple sclerosis.

3D voxels from DICOM data were modeled as a graph data structure on cloud infrastructure (Amazon). The graph included nodes which represent MRI voxels and the spatial relationships that exist between them. Nodes contained properties including a voxel's x, y, z coordinates as well as features such as signal intensities across modalities. Nodes were projected on a 3D grid using their coordinates for placement. Relationships between voxels model spatial neighborhoods in x, y, and z dimensions and across time.

Specifically, to represent MRI imaging data as a 3D graph, implicit information from a volumetric array was transformed into explicit relationships. Each voxel is a node (or vertex), with properties: each series, image, intensity, and relationships (edges, like neighbors in space and time). This analysis involved leveraging rich graph algorithms for spatial analysis to identify and analyze individual lesions and temporal analysis to track lesion development over time.

Nodes of lesions underwent thresholding both globally and locally for analyzing MRI lesions of patients. Specifically, given a 3D coordinate and threshold, a breadth first graph search (BFS) iterates through adjacent nodes while collecting nodes which satisfy the specified threshold condition [>, <, >=, <=,]. For example, the threshold condition may be a minimum voxel intensity. Nodes in a lesion community were given properties during initial modeling that were updated with results of global and local thresholding events. Lesion graph communities were updated based on established thresholds and integrated into temporal analysis of lesion evolution and disease activity.

Visual graph representation of MRI data revealed temporal progression of all lesions simultaneously. Lesions can be visually classified as consolidating/merging, expanding, or splitting across time using an interactive slider. Graph algorithms were used to establish multiple sclerosis disease activity including: lesion nodes, inter/intralesion relationships, lesion adjacency to neuroanatomy, intralesion voids (e.g., as a measure of permanent tissue damage), separated lesion surfaces from internal components, characterized lesions (e.g., lesion surface, texture, shape, topology, density, homogeneity), temporal changes (e.g., new lesion, enlarging lesion, or shrinking lesion), and volumetrics (e.g., total lesion load, merging, or splitting lesions).

Altogether, interactive 3D graph representations of MRI graph data augment traditional visualization and analysis by providing connectedness and temporal resolution into the disease process. Graphs highlight the connectedness of MRI data, the communities that compose structural features and disease processes, and the temporal relationships revealed during MS disease progression.

### Example 2: Methodology of Building a 3D Graph Representation of MRI Data from MS Patients

Described here is one example of building a 3D graph of the brain including individual nodes. Then, using the 3D graph of the brain, multiple lesions are identified using the iterative process of interrogating nodes for inclusion in node neighborhoods.

Specifically, the following brain scans are loaded:
a. 3D T1
b. FLAIR
c. A subtraction image Z-scored(FLAIR)−Zscored(T1)
d. An existing lesion mask (3D image, with values in array corresponding to lesion type)
e. Brain segmentation (with value corresponding to different brain regions)
f. Blank image (upon which to add new lesions)

The 3D graph is first constructed by loading the subtraction image, brain segmentation, and existing lesion mask into the graph. Here, the graph includes the following characteristics:
g. 1 node (e.g., vertex) per voxel
h. Each node has properties, which include the intensities from the subtraction image, value of the corresponding brain segmentation, and existing lesion mask
i. Each node has neighbors (eg. an “edge”), which are the nodes that are spatially next to the node (including diagonals, 26 total)

FIG. 8A depicts an example 3D graph with individual nodes that are connected to other nodes through connections.

Next, using the data (e.g., intensities) from the 3D T1 images and/or the FLAIR images captured from a subject, the presence of one or more lesions are identified in the 3D graph. First, a seed node for a lesion is identified. As one example, a user can add a seed node. As another example, the system identifies a likely seed node. The corresponding node (e.g., node corresponding to the seed node) within the subtraction image is identified and the intensity of that subtraction image is set as the “minimum threshold.”

Next, nodes adjacent to the seed node were interrogated for inclusion or exclusion from a node neighborhood based on whether the subtraction-image-intensity of that node is greater than or equal to the “minimum threshold”. This process is iterative until subsequent adjacent nodes no longer have intensity values that satisfy the minimum threshold. Thus, the nodes included in the node neighborhood are defined and the number of nodes is calculated. An example summary of the various lesions (e.g., as identified based on node neighborhoods) is shown in FIG. 8B. Specifically, the x,y,z coordinates correspond to a node's spatial location within the neighborhood, the “type” corresponds to the lesion type encoded within the “existing lesion mask”, and the count is the total number of nodes in the neighborhood.

FIG. 8C and FIG. 8D each shows the identification of a lesion within the brain. Specifically, FIG. 8C shows the identification of a lesion 820A within the brain defined by a node neighborhood based on a minimum threshold value 810A of 0.5. Additionally, FIG. 8D shows the identification of the lesion 820B using a different minimum threshold value 810B of −0.4. Here, lesion 820A and lesion 820B are the same lesion, but differently defined based on the use of different minimum thresholds. Given the lower minimum threshold value 810B of −0.4, a larger lesion 820B was identified. Conversely, given the higher minimum threshold value 810A of 0.5, a smaller lesion 820A was identified.

Further minimum thresholds were also applied for identifying the lesions. For example, as shown in FIGS. 8C and 8D, minimum threshold values of −0.3, −0.2, −0.1, 0, 0.1, 0.2, 0.3, and 0.4 were also applied to identify the node neighborhoods that define the lesion. Specifically, starting with the minimum threshold of 0.5 as shown in FIG. 8C, the minimum threshold was decremented by a set interval (e.g., 0.1) and the node neighborhood was recomputed. The size of the node neighborhood at that minimum threshold was computed. The process is then repeated for the next minimum threshold. Here, the process is repeated (e.g., decrement the minimum threshold, detect a new node neighborhood) until the neighborhood size is greater than a set level (e.g., 1000 nodes).

FIG. 8E depicts an example lesion community, lesion surface, and lesion shell that are defined using a 3D graph.

### Example 3: Use of 3D Graphs Developed from MRI Images of MS Patients

In this example the goal was to create a 3D visualization of an individual patient's brain (e.g., a MS patient's brain with MS lesions), in a way that is intuitive and familiar for physicians and patients. By displaying MRI images in a transformed 3D graph, physicians and patients can absorb the information faster and trust the metrics. Thus 3D graphs of MRI images provides utility for care and management of patients with MS. For example, 3D graphs of MRI images, by providing temporal and spatial analysis of a patient's MS, can be useful for differential diagnosis of the patient's MS, can be useful for selecting candidate therapies for the patient, and/or for determining an efficacy of therapies previously administered to the patient.

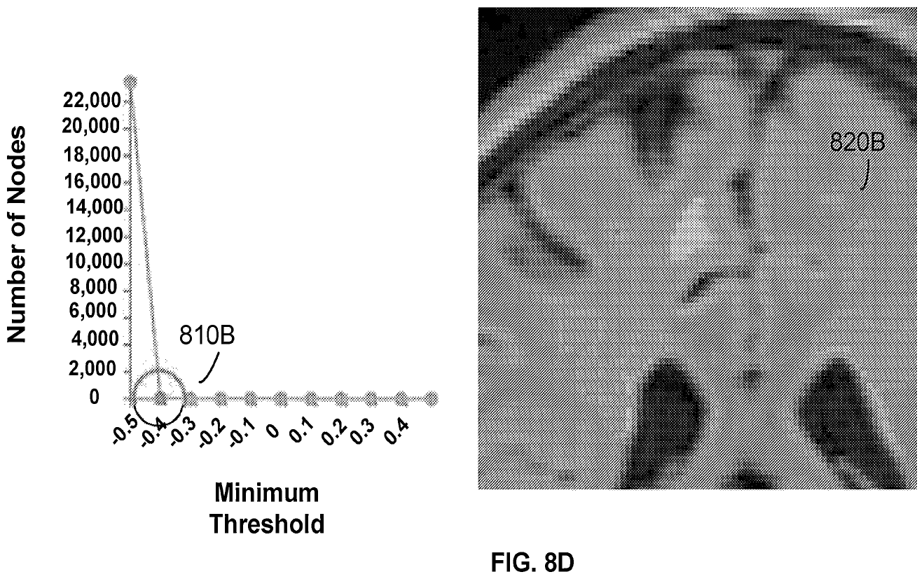

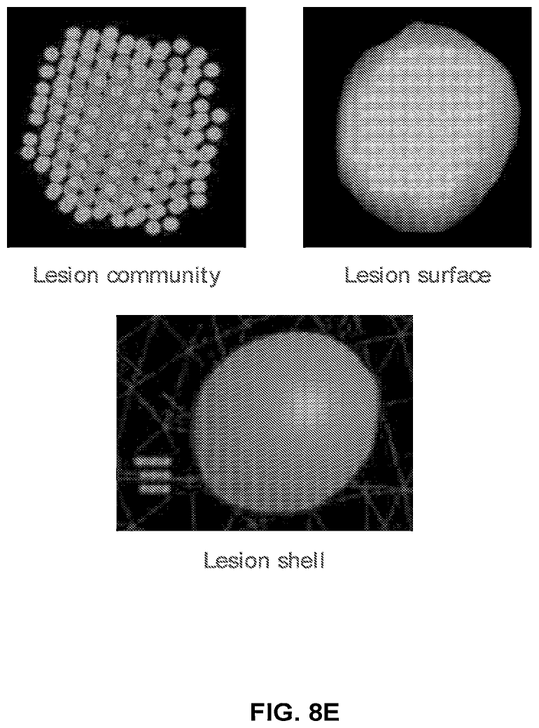

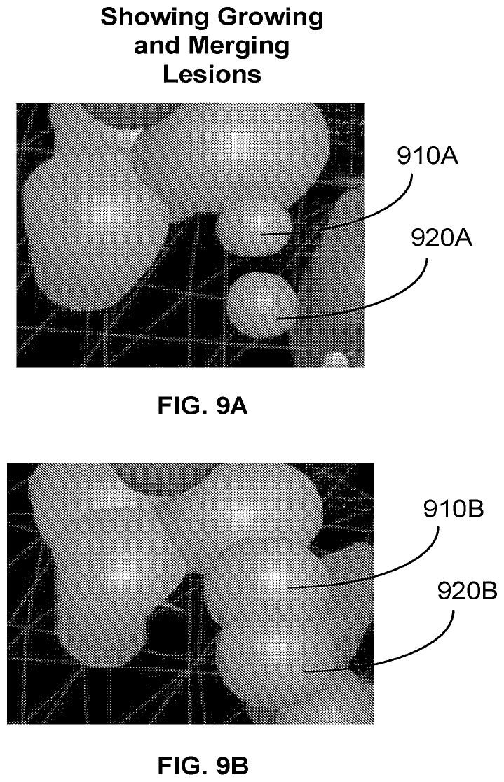

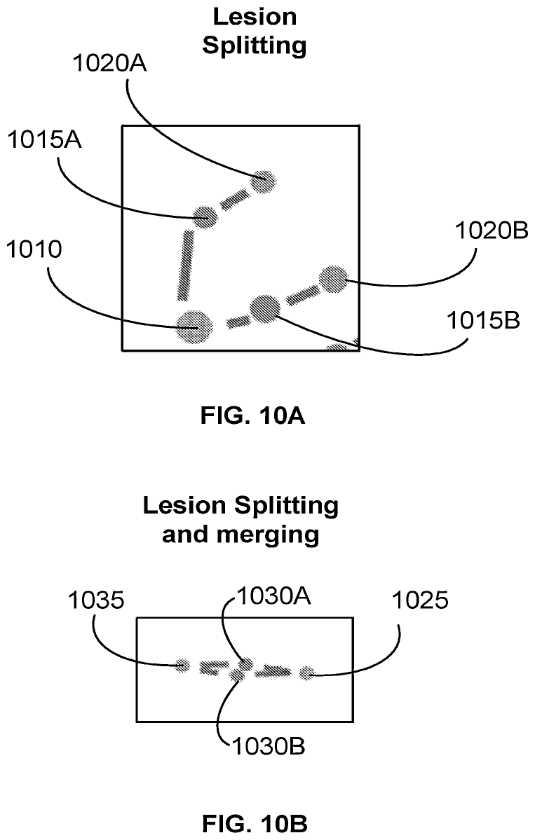

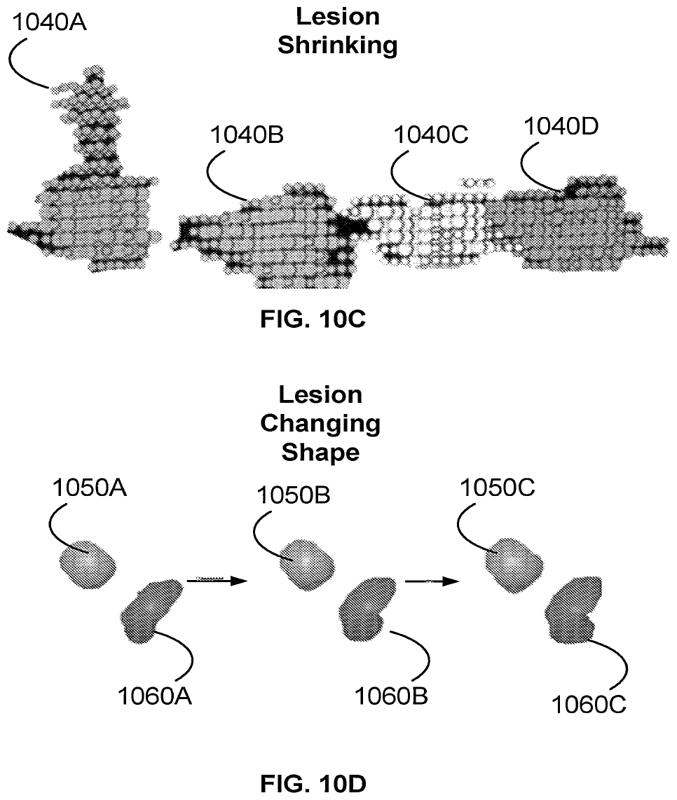

FIGS. 9A-9B and FIGS. 10A-10D show example multiple sclerosis lesions within a 3D graph that enables understanding of the temporal and spatial characteristics of a patient's MS. Thus, this understanding can guide the treatment care provided to the patient.

Specifically, FIGS. 9A and 9B depict the growing and merging of lesion bodies using a 3D graph. FIG. 9A shows identified lesions within the 3D graph for a set of images captured from a patient at a first timepoint. FIG. 9B shows identified lesions within the 3D graph for a set of images captured from the same patient at a second timepoint. Here, each of the lesions were identified as a node neighborhood using the methodology described herein. Lesion 910A shown in FIG. 9A increases in volumetric size to a larger lesion 910B. Additionally, lesion 920A shown in FIG. 9A similarly increases in volumetric size to a larger lesion 920B. Furthermore, lesion 910B and 920B are in contact with one another within the 3D graph shown in FIG. 9B, indicating the lesions 910B and 920B are merging as they are increasing in size. Here, the 3D graph transition from FIG. 9A to FIG. 9B indicates that the patient's multiple sclerosis is progressing. In a scenario in which the patient is undergoing treatment, the 3D graph transition from FIG. 9A to FIG. 9B can indicate that the treatment is lacking efficacy and therefore, a different treatment can be sought. Alternatively, if the patient has not yet undergone treatment, the 3D graph transition from FIG. 9A to FIG. 9B can indicate that the disease is progressing and therefore, a treatment is to be provided to the patient.

FIG. 10A depicts a lesion splitting within a 3D graph. Specifically, FIG. 10A shows a lesion (identified as a node neighborhood using the methodology described herein) and its progression across three different timepoints. At a first timepoint, the lesion 1010 is a single node neighborhood. At a second timepoint, the lesion has split into lesion 1015A and lesion 1015B which are represented by two separate neighborhoods. At a third timepoint, two separate lesions 1020A and 1020B are further observed.

FIG. 10B depicts a lesion splitting and merging within a 3D graph. Specifically, FIG. 10B shows a lesion (identified as a node neighborhood using the methodology described herein) and its progression across three different timepoints. At a first timepoint, the lesion 1025 is a single node neighborhood. At a second timepoint, the lesion has split into lesion 1030A and lesion 1030B which are represented by two separate node neighborhoods. At a third timepoint, the separate lesions have merged again into a single lesion 1035.

FIG. 10C depicts a shrinking lesion within a 3D graph. Specifically, FIG. 10C shows a lesion (identified as a node neighborhood using the methodology described herein) and its progression across four different timepoints. At a first timepoint, the lesion 1040A is represented by a single node neighborhood. In comparison to the lesion 1040A, the lesion at the second timepoint (e.g., lesion 1040B), third timepoint (lesion 1040C), and fourth timepoint (lesion 1040D) are smaller in volumetric size. Here, the size of the lesion at a particular timepoint is determined according to the nodes (e.g., number of nodes) included in the node neighborhood that defines the lesion. Altogether, the 3D graph including the lesion shown in FIG. 10C indicates that the patient's lesion is shrinking. In a scenario in which the patient is undergoing treatment, the 3D graph shown in FIG. 10C can indicate that the treatment is effective. In this scenario, the treatment can continue to be provided to the patient.

FIG. 10D depicts a changing shape of a lesion within a 3D graph. Specifically, FIG. 10D shows two lesions (each of which is identified as a node neighborhood using the methodology described herein) and their progression across three different timepoints. Lesion 1050A and lesion 1060A are shown in the 3D graph (left panel) at a first timepoint. Lesion 1050B and lesion 1060B are next shown in the 3D graph (middle panel) at a second timepoint. Lesion 1050C and lesion 1060C are next shown in the 3D graph (right panel) at a third timepoint. Here, one of the lesions remains largely unchanged across all three timepoints (see lesion 1050A, lesion 1050B, and lesion 1050C). Thus, this lesion can be categorized as a stable lesion that is unchanging over time. In contrast, the second lesion exhibits a change in topology, as indicated by the increasing curvature in the lesion over time (see lesion 1060A, lesion 1060B, and lesion 1060C).

What is claimed is:

1. A method comprising:
obtaining a set of images captured from an individual, the set of images comprising an anatomical abnormality;
generating a three dimensional (3D) graph using the set of images, the 3D graph comprising a plurality of nodes representing voxels and the anatomical abnormality;
establishing a seed node in the 3D graph indicative of a presence of the anatomical abnormality, the seed node defined by an initial voxel coordinate;
defining a node neighborhood comprising the seed node indicative of a 3D volume of the anatomical abnormality by:
iteratively interrogating one or more adjacent nodes for inclusion or exclusion from the node neighborhood, wherein the interrogation of each of the one or more adjacent nodes is based on an intensity value of the adjacent node and an anatomical location of the adjacent node;
generating a representation of the 3D volume of the anatomical abnormality; and
storing at least the representation of the 3D volume of the anatomical abnormality.

2. The method of claim 1, further comprising:
obtaining a second set of images captured from the individual, the second set of images further comprising the anatomical abnormality;
generating a second three dimensional (3D) graph using the second set of images, the 3D graph comprising a plurality of nodes representing voxels and the anatomical abnormality;
establishing a seed node in the second 3D graph indicative of a presence of the anatomical abnormality in the second 3D graph, the seed node defined by an initial voxel coordinate;
defining a node neighborhood comprising the seed node indicative of a 3D volume of the anatomical abnormality by:
iteratively interrogating one or more adjacent nodes for inclusion or exclusion from the node neighborhood, wherein the interrogation of each of the one or more adjacent nodes is based on an intensity value of the adjacent node and an anatomical location of the adjacent node;
generating a second representation of the 3D volume of the anatomical abnormality;
retrieving at least the stored representation of the 3D volume of the anatomical abnormality;
characterizing the anatomical abnormality by comparing the stored representation of the 3D volume of the anatomical abnormality to the second representation of the 3D volume of the anatomical abnormality.

3. The method of claim 1, wherein interrogation of the one or more adjacent nodes of the 3D graph or of the second 3D graph comprises:
retrieving a threshold value previously determined for the anatomical location;
comparing the intensity value of the adjacent node to the retrieved threshold value.

4. The method of claim 3, further comprising:
responsive to determining that the intensity value of the adjacent node exceeds the retrieved threshold value, including the adjacent node in the node neighborhood.

5. The method of claim 3, further comprising:
responsive to determining that the intensity value of the adjacent node is less than the retrieved threshold value, excluding the adjacent node from the node neighborhood.

6-31. (canceled)

32. A non-transitory computer readable medium comprising instructions that, when executed by a processor, cause the processor to:
obtain a set of images captured from an individual, the set of images comprising an anatomical abnormality;
generate a three dimensional (3D) graph using the set of images, the 3D graph comprising a plurality of nodes representing voxels and the anatomical abnormality;
establish a seed node in the 3D graph indicative of a presence of the anatomical abnormality, the seed node defined by an initial voxel coordinate;
define a node neighborhood comprising the seed node indicative of a 3D volume of the anatomical abnormality by:
iteratively interrogate one or more adjacent nodes for inclusion or exclusion from the node neighborhood, wherein the interrogation of each of the one or more adjacent nodes is based on an intensity value of the adjacent node and an anatomical location of the adjacent node;
generate a representation of the 3D volume of the anatomical abnormality; and
store at least the representation of the 3D volume of the anatomical abnormality.

33-62. (canceled)
